(19) United States
(12) Patent Application Publication
(10) Pub. No.: US 2022/0000360 A1
(43) Pub. Date: Jan. 6, 2022

(54) METHOD AND SYSTEM FOR PERFORMING INTELLIGENT REFRACTIVE ERRORS DIAGNOSIS

(71) Applicant: Zaaf Science Group LLC, Newington, CT (US)

(72) Inventor: Yan Zhang, Newington, CT (US)

(21) Appl. No.: 17/364,304

(22) Filed: Jun. 30, 2021

Related U.S. Application Data
(63) Continuation-in-part of application No. 17/364,258, filed on Jun. 30, 2021.
(60) Provisional application No. 63/046,715, filed on Jul. 1, 2020.
(60) Provisional application No. 63/107,392, filed on Oct. 29, 2020.

(51) Int. Cl. A61B 3/032; A61B 3/00

(52) U.S. Cl. CPC A61B 3/032; A61B 3/0033

(57) ABSTRACT

There is provided a method and a system for intelligently determining an eyeglass prescription of a patient. The method, executed by a processor, includes: obtaining patient information from the patient to generate initial sphere, cylinder, axis, add, and prism values; performing measurements to generate at least one updated sphere, cylinder, axis, add, and prism value based on communication with the patient; repeating the performing measurements to generate optimized sphere, cylinder, axis, add, and prism values; and outputting the optimized sphere, cylinder, axis, add, and prism values to a database.

### CROSS-REFERENCE TO RELATED PATENT APPLICATIONS

This application claims priorities from non-provisional application Ser. No. 17/364,258 filed Jun. 30, 2021, from provisional application No. 63/046,715, filed Jul. 1, 2020, and from provisional application No. 63/107,392, filed Oct. 29, 2020, the content of which are incorporated herein in the entirety by references.

### TECHNICAL FIELD

The present disclosure relates to the field of optometry, and more particularly relates to a method and system for performing intelligent refractive errors diagnosis automatically.

### BACKGROUND

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference is individually incorporated by reference.

A phoropter is a refraction device used during an eye examination to determine the refractive errors of a patient including, for example, spherical, cylinder, axis (i.e. axis angle of the cylinder), prism amplitude, prism base direction, and/or add power values. Conventionally, major components of the phoropter include a set of spherical and cylindrical lenses, filtered lenses, prisms, a JCC (Jackson Cross-Cylinder) used for astigmatism measurement, a reading rod, a card holder, a near point reading card, apertures for the left eye and right eye, etc. The output of a refraction examination may be optimized refractive error values, and may be used to determine the patient's eyeglass prescription.

Currently, doctors rely on the phoropter to diagnose the patient's refractive error and then provide to the patient a corresponding eyeglass prescription. Typically, the doctor uses the previous eyeglass prescription of the patient or data from an auto-refractor test as a starting point to start the refraction. An auto-refractor may be used to measure the refractive error of the patient's eyes without input from the patient. However, the auto-refractor often does not output an accurate prescription for a variety of reasons, including (1) the patient may not look directly at the target provided by the auto-refractor; (2) the patient may have dry eye, resulting in a broken tear film layer; (3) the patient may display proximal accommodation when viewing the target displayed by the auto-refractor; (4) the patient's pupil may be too small for the auto-refractor to conduct an accurate analysis. Hence, the data from the auto-refractor typically is not used to conduct the entirety of a refraction exam, but rather utilized as a starting point for the exam.

FIG. 1 illustrates a flowchart showing a conventional system for conducting a refraction exam. As shown in FIG. 1, optometrist 102 acts as an interface between refracting device 106 (e.g. the phoropter) and patient 104. Optometrist 102 typically presents the patient with multiple views through the phoropter by adjusting certain optical and/or mechanical elements inside refracting device 106 such as, without limitation, the spherical lens and/or the cylinder lens. Patient 104 is given time to examine the target through the aperture of refracting device 106, and optometrist 102 asks the patient to compare the views to determine which view is perceived to be clearer. Based on the comparison, subsequent rounds of adjusting elements of refracting device 106 and presenting multiple views to the patient are conducted. At the end of refraction process, patient 104 may be asked to read out letters, words, symbols or images shown on a screen. Different sized letters correspond to the different levels of visual acuity. Optometrist 102 then may document results including, but not limited to, spherical, cylinder, axis, prism amplitude, base direction, and add power. Based on the results, patient 104 may receive an eyeglass prescription from optometrist 102.

Another type of refracting device is a pair of liquid lenses properly mounted in a mechanical structure such as a trial frame, wherein the patient looks through the liquid lenses during a refraction examination. The surface shapes of the liquid lenses are changeable, leading to different spherical, cylinder and prism power combinations. With a proper combination of spherical, cylinder, axis, and prism values, the patient can achieve improved vision, typically 20/20 in a healthy individual.

Additionally, a refracting device may be a set of liquid crystal lenses mounted in a mechanical structure such as a trial frame wherein the patient looks through the liquid crystal lenses during the refraction examination. Properties of the liquid crystal lenses, such as surface shape, can be changed, resulting in different spherical, cylinder and prism power combinations. With the proper combination of cylinder, spherical, and prism powers, the patient can achieve improved vision, typically 20/20 in a healthy individual.

As used herein, refracting device refers to a device or a set of devices which can be used to find the optimal eye glass prescription of the patient by varying its optical and/or mechanical components' settings and or properties. For example, without limitation, virtual reality glasses may also be refracting devices.

There may be many types of refracting devices which can be used during refraction examination, wherein a refraction examination may be one component of an eye examination. The common features of the aforementioned refracting devices are that they can change the spherical, cylinder, prism values of their lenses separately or in combination to achieve optimal vision for the patient. During a traditional eye examination, the patient looks through optical components mounted in the refracting device. The patient is presented with two views or images, and the patient will determine which view or image looks better. The patient then tells the optometrist his/her choices. The optometrist will modify the spherical, cylinder, prism power separately or in combination and continue the above cycle.

There is a need to perform autonomous refraction with minimal doctor supervision or participation, or even without local doctor supervision/participation due to (1) avoiding transmission of disease between a doctor and a patient; (2) increasing exam efficiency; (3) reducing overall health care cost, and (4) providing convenience to the patient.

As will be appreciated by one skilled in the art, various professionals may perform an eye examination, and may not be limited to only optometrists. For example, without limitation, a Doctor of Medicine (MD), Doctor of Osteopathic Medicine (DO), etc.

Therefore, a heretofore unaddressed need exists in the art to address the aforementioned deficiencies and inadequacies.

### SUMMARY

In one embodiment, an automated refraction process for determining an eyeglass prescription of a patient, executed by a processor, is provided. The process includes obtaining patient information from the patient to generate initial sphere, cylinder, axis, add, and prism values; performing measurements to generate at least one updated sphere, cylinder, axis, add, and prism value based on communication with the patient; repeating the performing measurements to generate optimized sphere, cylinder, axis, add, and prism values; and outputting the optimized sphere, cylinder, axis, add, and prism values to a database.

In another embodiment, performing measurements includes: performing sphere measurements, cylinder measurements, axis measurements, add measurements, and prism measurements to obtain the optimized sphere, cylinder, axis, add, and prism values.

In another embodiment, the process further includes: sending an error report upon receiving an error; and sending a completion report upon completion of the automated refraction process.

In another embodiment, the performing cylinder measurements includes: setting the initial cylinder value to a reference cylinder value; applying the initial cylinder value to select a first optic and a second optic; determining via a first patient input that the first optic is perceived by the patient to be clearer than the second optic, thus yielding an updated cylinder value from the initial cylinder value, while maintaining a spherical equivalent; assigning the updated cylinder value to the initial cylinder value; and repeating the applying, the determining, and the assigning to yield an intermediate cylinder value.

In another embodiment, the initial cylinder value is greater than a first threshold value.

In another embodiment, the process, further includes: generating a cylinder difference from the reference cylinder value and the intermediate cylinder value; verifying that the cylinder difference is greater than a second threshold value; selecting a third optic using the reference cylinder value and a fourth optic using the intermediate cylinder value; determining via a second patient input whether the third optic is perceived by the patient to be clearer than the fourth optic; and generating the optimized cylinder value based on a result of the determining, while maintaining the spherical equivalent.

In another embodiment, performing measurements to generate the updated axis value includes: setting the initial axis value to a reference axis value; applying the initial axis value to select a fifth optic and a sixth optic; determining via a third patient input that the fifth optic is perceived by the patient to be clearer than the sixth optic, thus yielding an updated axis value from the initial axis value; assigning the updated axis value to the initial axis value; and repeating the applying, the determining, and the assigning to yield an intermediate axis value.

In another embodiment, the process, further includes: generating an axis difference from the reference axis value and the intermediate axis value; verifying that the axis difference is greater than a third threshold value; selecting a seventh optic using the reference axis value and an eighth optic using the intermediate axis value; determining via a fourth patient input whether the seventh optic is perceived by the patient to be clearer than the eighth optic; and generating the optimized axis value based on a result of the determining.

In another embodiment, the performing sphere measurements comprises: selecting a first letter size of a first set of letters to show the patient; generating an updated sphere value by adjusting the initial sphere value to improve the patient's perception of the first set of letters; and generating a tag value based on the first letter size.

In another embodiment, the process, further includes: selecting a second letter size based on the tag value; displaying a line of a second set of letters of second letter size to the patient; recording a response from the patient; and adjusting the updated sphere value based on the response to generate an optimized sphere value.

In another embodiment, the first set of letters and second set of letters is a set of words or a set of images.

In another embodiment, the process further includes: adjusting the optimized sphere value for a left eye and a right eye of the patient independently such that a first view presented to the left eye is visually identical to a second view presented to the right eye.

In another embodiment, the performing prism measurements includes: applying the initial prism value to select a ninth optic; determining via a fifth patient input that the ninth optic is perceived by the patient to be unclear, thus yielding an updated prism value from the initial prism value; assigning the updated prism value to the initial prism value; and repeating the applying, the determining, and the assigning to yield an optimized prism value.

In another embodiment, the process further includes: recording a baseline speaking time of the patient; recording a speaking speed of the patient; comparing the baseline speaking time with the speaking speed to determine a confidence level; and utilizing the confidence level to calculate a correction rate during said performing.

In another embodiment, the performing add measurements includes: actuating a motor to set an automated reading rod into an active position to display a line of letters to the patient; recording a response from the patient; adjusting the updated add value based on the response to generate an optimized add value, wherein the updated add value is based on a reference chart and the reference chart is stored on the database; and actuating the motor to set the automated reading rod into an inactive position.

In another embodiment, the initial sphere, cylinder, axis and prism values are chosen from the group consisting of a current eyeglass prescription of the patient, a last eyeglass prescription on file of the patient, and auto refractor data.

In another embodiment, the initial sphere value is greater than a threshold value and the threshold value is calculated based on the patient information.

In another embodiment, the process further includes: communicating with the patient via a patient input device, wherein the patient input device is selected from a group consisting of a joystick, a keyboard, a touchscreen device, a camera, and a microphone.

In another embodiment, the communicating uses voice recognition to record a response from the patient.

In another embodiment, a system is provided. The system includes: a processor; and a memory that contains instructions that are readable by said processor to cause said processor to perform actions of: obtaining patient information from the patient to generate initial sphere, cylinder, axis and prism values; performing measurements to generate at least one updated sphere, cylinder, axis, and prism value based on communication with the patient; repeating the performing measurements to generate optimized sphere, cylinder, axis, and prism values; and outputting the optimized sphere, cylinder, axis, and prism values to a database.

### BRIEF DESCRIPTION OF THE DRAWINGS

The accompanying drawings illustrate one or more embodiments of the present disclosure and, together with the written description, serve to explain the principles of the present disclosure, wherein:

FIG. 1 illustrates a flowchart showing a conventional system for conducting a refraction exam;

FIG. 2 illustrates a block diagram of an exemplary system for conducting an automated refraction process

FIG. 3 illustrates a block diagram showing an exemplary secondary system for performing an automated refraction process, in accordance with an embodiment of the present disclosure;

FIGS. 4A-4C illustrate exemplary systems for conducting a refraction exam, wherein FIG. 4A shows a first system, FIG. 4B shows a second system, and FIG. 4C shows a third system, in accordance with an embodiment of the present disclosure;

FIGS. 5A-5D illustrate exemplary automated refraction systems, wherein FIG. 5A shows a first system, FIG. 5B shows a second system, FIG. 5C shows a third system, and FIG. 5D shows a fourth system, in accordance with an embodiment of the present disclosure;

FIGS. 6A-6F illustrate exemplary user interfaces of an automated refraction system, wherein FIG. 6A shows a first interface, FIG. 6B shows a second interface, FIG. 6C shows a third interface, FIG. 6D shows a fourth interface, FIG. 6E shows a fifth interface, and FIG. 6F shows a sixth interface, in accordance with an embodiment of the present disclosure;

FIG. 7 illustrates a flowchart showing an automated refraction process, in accordance with an embodiment of the present disclosure;

FIG. 8 illustrates a flowchart showing an exemplary data reconciliation module, in accordance with an embodiment of the present disclosure;

FIG. 9 illustrates a flowchart showing an exemplary sanity check module, in accordance with an embodiment of the present disclosure;

FIG. 10 illustrates a flowchart showing an exemplary first voice integration module, in accordance with an embodiment of the present disclosure;

FIG. 11 illustrates a flowchart showing an exemplary second voice integration module, in accordance with an embodiment of the present disclosure;

FIG. 12 illustrates a flowchart showing an exemplary 20/60 line clear module, in accordance with an embodiment of the present disclosure;

FIG. 13 illustrates a flowchart showing an exemplary speaking speed measurement module, in accordance with an embodiment of the present disclosure;

FIG. 14 illustrates a flowchart showing an exemplary 20/40 line clear module, in accordance with an embodiment of the present disclosure;

FIG. 15 illustrates a flowchart showing an exemplary cylinder (cyl) search module, in accordance with an embodiment of the present disclosure;

FIG. 16 illustrates a flowchart showing an exemplary axis module, in accordance with an embodiment of the present disclosure;

FIG. 17 illustrates a flowchart showing an exemplary alternative axis module, in accordance with an embodiment of the present disclosure;

FIGS. 18A-18B illustrate flowcharts showing an exemplary cyl module, wherein FIG. 18A shows a cyl module and FIG. 18B shows a process for maintaining a spherical equivalent, in accordance with an embodiment of the present disclosure;

FIGS. 19A-19B illustrate flowcharts showing alternative cyl and axis paths, wherein FIG. 19A shows a first alternative path and FIG. 19B shows a second alternative path in accordance with an embodiment of the present disclosure;

FIG. 20 illustrates a flowchart showing an exemplary axis compare module, in accordance with an embodiment of the present disclosure;

FIG. 21 illustrates a flowchart showing an exemplary cyl compare module, in accordance with an embodiment of the present disclosure;

FIG. 22 illustrates a flowchart showing an exemplary optimization at 20/40 module, in accordance with an embodiment of the present disclosure;

FIG. 23 illustrates a flowchart showing an exemplary optimization at 20/60 module, in accordance with an embodiment of the present disclosure;

FIG. 24 illustrates a flowchart showing an exemplary read 20/20 size module, in accordance with an embodiment of the present disclosure;

FIG. 25 illustrates a flowchart showing an exemplary large size module, in accordance with an embodiment of the present disclosure;

FIG. 26 illustrates a flowchart showing an exemplary binocular balance module, in accordance with an embodiment of the present disclosure;

FIG. 27 illustrates a flowchart showing an exemplary near vision test module, in accordance with an embodiment of the present disclosure;

FIG. 28 illustrates a flowchart showing an exemplary automated refraction process, in accordance with an embodiment of the present disclosure;

FIGS. 29A-29E illustrate exemplary views to be shown to a patient, wherein FIG. 29A shows a first view, FIG. 29B shows a second view, FIG. 29C shows a third view, FIG. 29D shows a fourth view, and FIG. 29E shows a fifth view, in accordance with an embodiment of the present disclosure;

FIG. 30 illustrates a flowchart showing presentation of different views to a patient, in accordance with an embodiment of the present disclosure;

FIGS. 31A-31D illustrate exemplary patterns to be shown to a patient, wherein FIG. 31A shows a first pattern, FIG. 31B shows a second pattern, FIG. 31C shows a third pattern, and FIG. 31D shows a fourth pattern, in accordance with an embodiment of the present disclosure;

FIG. 32 illustrates a flowchart showing a second voice recognition process, in accordance with an embodiment of the present disclosure; and

FIG. 33 illustrates a flowchart showing a transition from autonomous refraction examination to non-autonomous refraction examination, in accordance with an embodiment of the present disclosure.

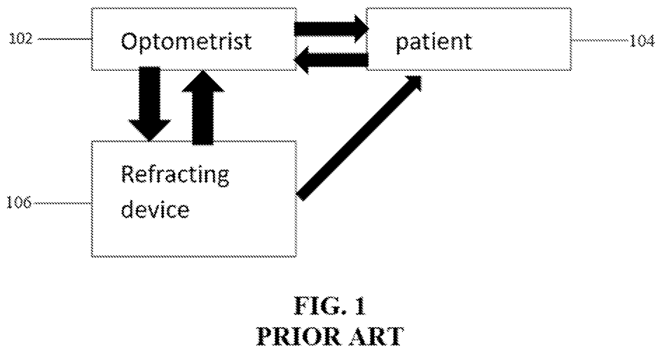

### DETAILED DESCRIPTION

The present disclosure will now be described more fully hereinafter with reference to the accompanying drawings, in which exemplary embodiments of the present disclosure are shown. The present disclosure may, however, be embodied in many different forms and should not be construed as limited to the embodiments set forth herein. Rather, these embodiments are provided so that this disclosure is thorough and complete, and will fully convey the scope of the present disclosure to those skilled in the art. Like reference numerals refer to like elements throughout.

The terms used in this specification generally have their ordinary meanings in the art, within the context of the present disclosure, and in the specific context where each term is used. Certain terms that are used to describe the present disclosure are discussed below, or elsewhere in the specification, to provide additional guidance to the practitioner regarding the description of the present disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It is appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the present disclosure or of any exemplified term. Likewise, the present disclosure is not limited to various embodiments given in this specification.

It is understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It is understood that, although the terms firstly, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present disclosure.

It is understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It is also appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the present disclosure. As used herein, the singular forms “a,” “an,” and “the” are intended to include the multiple forms as well, unless the context clearly indicates otherwise. It is further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It is understood that relative terms are intended to encompass different orientations of the device in addition to the orientation shown in the figures. For example, if the device in one of the figures is turned over, elements described as being on the “lower” side of other elements will then be oriented on the “upper” sides of the other elements. The exemplary term “lower” can, therefore, encompass both an orientation of lower and upper, depending on the particular orientation of the figure. Similarly, for the terms “horizontal”, “oblique” or “vertical”, in the absence of other clearly defined references, these terms are all relative to the ground. Similarly, if the device in one of the figures is turned over, elements described as “below” or “beneath” other elements will then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present disclosure belongs. It is further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially,” “generally” or “approximately” shall generally mean within 20 percent, preferably within 10 percent, and more preferably within 5 percent of a given value or range. Numerical quantities given herein are approximate, meaning that the terms “around,” “about,” “substantially,” “generally” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the present disclosure.

Embodiments of the present disclosure are illustrated in detail hereinafter with reference to accompanying drawings. It should be understood that specific embodiments described herein are merely intended to explain the present disclosure, but not intended to limit the present disclosure.

In order to further elaborate the technical means adopted by the present disclosure and its effect, the technical scheme of the present disclosure is further illustrated in connection with the drawings and through specific mode of execution, but the present disclosure is not limited to the scope of the implementation examples.

The present disclosure relates to the field of optometry, and more particularly relates to a method and system for performing intelligent refractive errors diagnosis.

As used herein, the letters shown on the display can be described according to size. For example: a 20/20 size letter at 6 meters away from a patient has a height of 8.75 mm, and a 20/200 size letter at 6 meter away from a patient has a height of 87.5 mm.

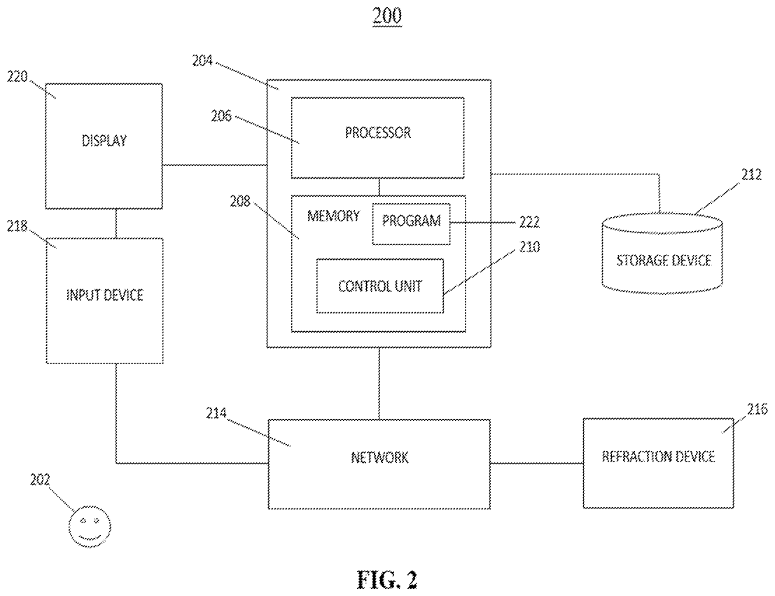

FIG. 2 illustrates a block diagram of an exemplary system for conducting an automated refraction process.

System 200 includes a computer 204 coupled to a network 214, a storage device 212, and a refraction device 216.

Network 214 is a data communications network. Network 214 may be a private network or a public network, and may include any or all of (a) a personal area network, e.g., covering a room, (b) a local area network, e.g., covering a building, (c) a campus area network, e.g., covering a campus, (d) a metropolitan area network, e.g., covering a city, (e) a wide area network, e.g., covering an area that links across metropolitan, regional, or national boundaries, (f) the Internet, or (g) a telephone network. Communications are conducted via network 214 by way of electronic signals and optical signals. Additionally, the devices of system 200 may communicate via wired connection.

Computer 204 includes a processor 206 and a memory 208 coupled to processor 206. Although computer 204 is represented herein as a standalone device, it is not limited to such, but instead can be coupled to other devices (not shown) in a distributed processing system. For example, computer 204 is coupled to an input device 218 and a display 220.

Processor 206 is an electronic device configured of logic circuitry that responds to and executes instructions.

Memory 208 is a tangible computer-readable storage medium encoded with a computer program. In this regard, memory 208 stores files and instructions, for example, a program 222, that are readable and executable by processor 206 for controlling the operation of processor 206. Memory 208 can be implemented in a random access memory (RAM), a hard disc drive, solid-state drive, a read only memory (ROM) or a combination thereof. Memory 208 includes a program 222.

Storage device 212 stores a plurality of programs and subprograms. Storage device 212 stores the plurality of programs and subprograms in accordance with the functions inside the plurality of programs and subprograms. It also stores information of usage record of the plurality of programs and subprograms.

Storage device 212 may be implemented in any form of storage device. Storage device 212 may be implemented as separate components, e.g., two separate hard drives, or in a single physical component, e.g., a single database.

Input device may be, for example, without limitation, a keyboard, speech recognition subsystem or gesture recognition subsystem, etc. for enabling a user 202 to communicate information via network 214, to and from computer 204. A cursor control or a touch-sensitive screen may be included in input device 218 to allow user 202 to communicate additional information and command selections to processor 206 and computer 204.

Processor 206 outputs, to storage device 212, a result of an execution of the method described herein.

In the present disclosure, although operations are described as being performed by computer 204, or by system 200 or its subordinate systems, the operations are actually performed by processor 206.

Additional devices, such as refraction device 216, may communicate with computer 204 via network 214. Refraction device 216 may be used to conduct a refraction examination. Input from user 202 inputted via input device 218 in response to prompts from refraction device 216 may be sent to control unit. In turn, control unit 210 may communicate with processor 206 according to program 222 in generating hardware adjustment instructions to be sent to refraction device 216 via network 214. Additionally, control unit 210 may generate instructions to be displayed to user 202 via display 220.

Display 220 may alternatively be an output device. The output device may include one or more of, for example, without limitation, a speaker, printer, etc.

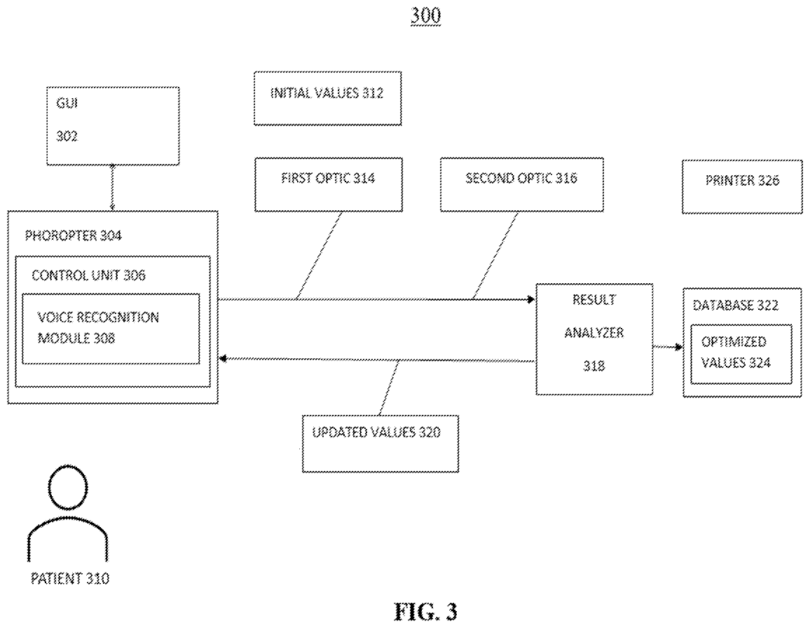

FIG. 3 illustrates a block diagram showing an exemplary secondary system for performing an automated refraction process, in accordance with an embodiment of the present disclosure.

System 300 includes phoropter 304 in communication with GUI 302, initial values 312, first optic 312, second optic 314, printer 326, result analyzer 314, updated values 320, and database 318.

System 300 may include control unit 306 incorporated within phoropter 304, and may perform a refraction examination process independently, in combination with a separate system (e.g. system 200), and/or with, for example, without limitation, a doctor, optometrist, technician, assistant, etc.

The doctor, optometrist, technician, assistant, etc. may interact with phoropter 304 via graphical user interface (GUI) 302. Phoropter 304 may also be controlled via control unit 306. Although control unit 306 is depicted as being integrated in phoropter 304, as will be appreciated by one skilled in the art, control unit 306 may also be external to phoropter 304. Control unit 306 may be, for example, without limitation, a computer, mobile phone, tablet, etc. Control unit 306 may interact with patient 310 via voice recognition module 308.

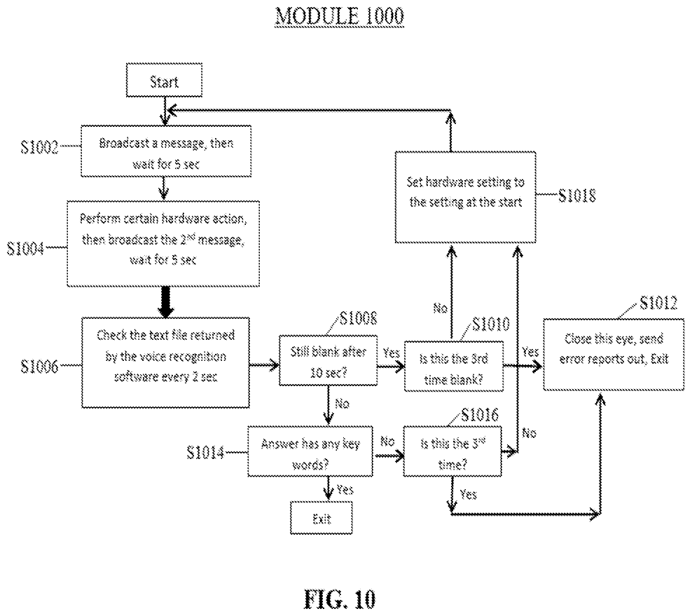

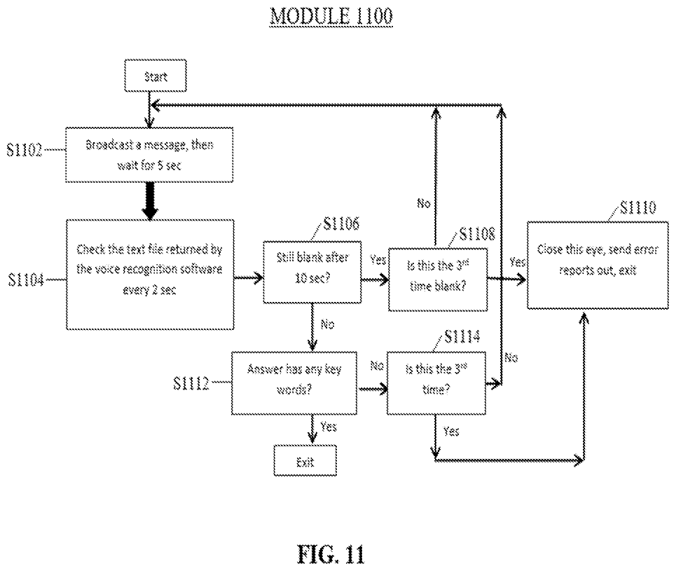

Voice recognition module 308 may utilize a first voice recognition process as will be described below with reference to FIG. 10 and a second voice recognition process as will be described below with reference to FIG. 11 to receive input from the user. Alternative input means may also be used to receive input from the user, such as, without limitation, a keyboard, joystick, etc.

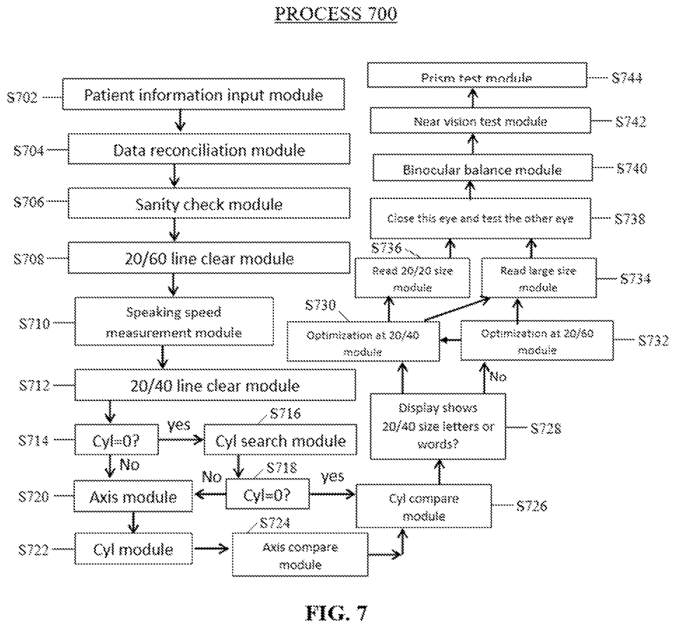

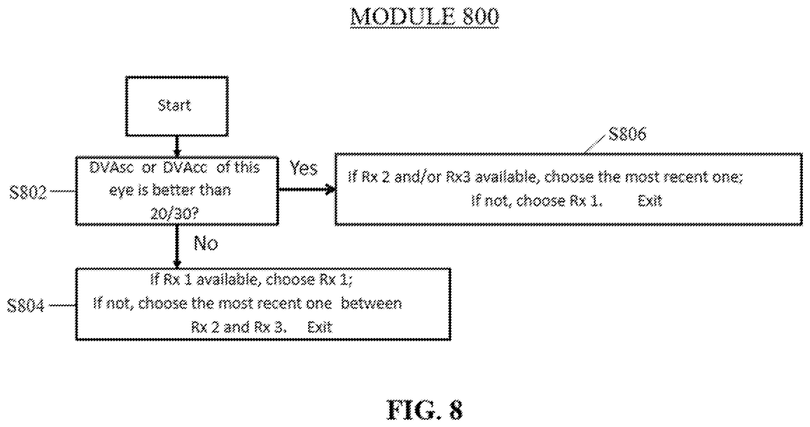

Phoropter 304 may be configured to generate first optic 314 and second optic 316 based on initial values 312. First optic 314 may be a particular set of hardware configurations of phoropter 304 to present a first view to user 310, while second optic view 316 may be a particular set of hardware configurations of phoropter 304 to present a second view presented to user 310. For example, without limitation, initial values 312 may be reconciled based on information obtained from patient 310. The information obtained from patient 310 may be a starting point for the automated refraction process. The information obtained from patient 310 may include an initial sphere value, an initial cylinder value, an initial axis value, an initial prism value, an initial add value, etc. Initial values 312 may be sourced from one or more of a current eyeglass prescription of the patient, a last eyeglass prescription on file of the patient, and auto refractor data. If the patient information includes more than one potential starting point (i.e. the current eyeglass prescription of the patient, the last eyeglass prescription on file of the patient, and the auto refractor data), initial values 312 may be reconciled from the patient information via a data reconciliation module, as will be described below with reference to FIG. 8. Once initial values 312 are determined, phoropter 304 may be adjusted based on initial values 312 to generate first optic 314 and second optic 316. Patient 310 may be shown the first view using first optic 314 with a first sph value and the second view using second optic 316 with hardware settings of +1.00 D more than the first sph value. The patient may respond indicating which view is perceived to be clearer, and the response may be recorded by voice recognition module 308. The response may be analyzed via result analyzer 318. If needed, result analyzer 318 may send updated values 320 to phoropter 304 such that control unit 306 may adjust the hardware settings of phoropter 304 according to updated values 320, thus updating first optic 314 and second optic 316. If the automated refraction process is completed, optimized values 318 of the automated refraction process may be stored on database 316, wherein optimized values 322 may be a final eyeglass prescription of patient 310. In one embodiment, the final eyeglass prescription may be output using printer 326. Additionally, a completion report including a completion alert may be sent out to the appropriate staff for review.

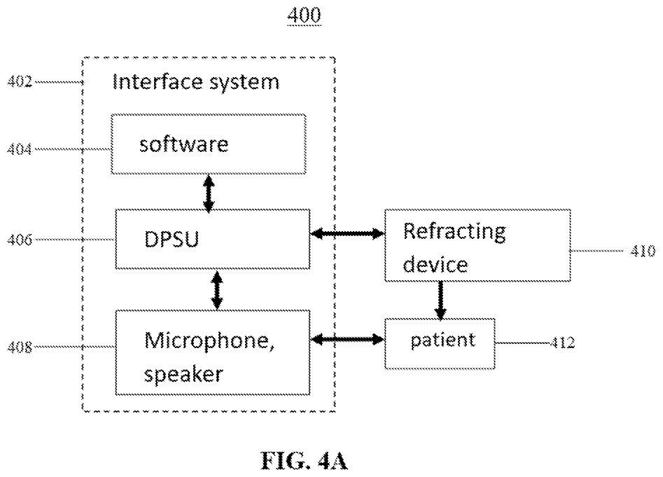

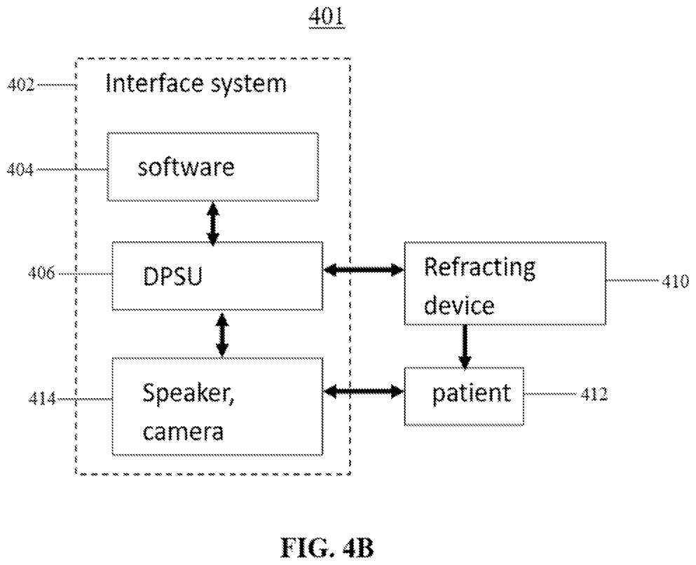

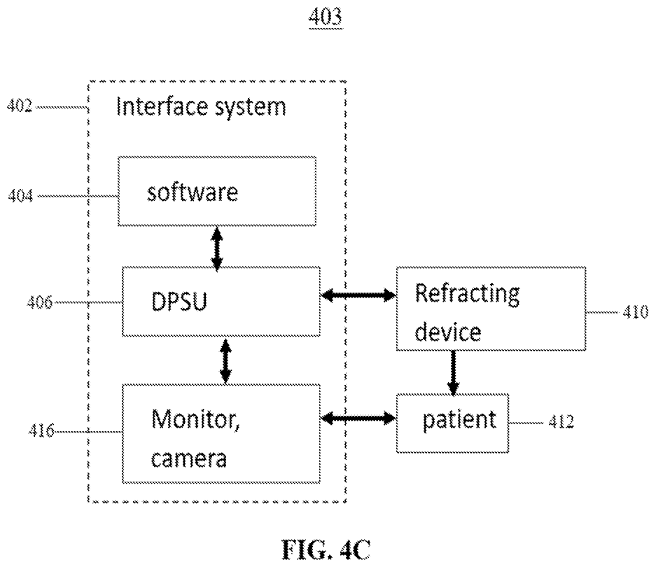

FIGS. 4A-4C illustrate exemplary systems for conducting a refraction exam, wherein FIG. 4A shows a first system, FIG. 4B shows a second system, and FIG. 4C shows a third system, in accordance with an embodiment of the present disclosure.

System 400 includes interface system 402 and refracting device 410, wherein interface system includes software 404, data processing and storage unit (DPSU) 406, and microphone and speaker 408.

Interface system 402 acts as the interface between patient 410 and refracting device 412. Interface system 402 includes, for example, without limitation: DPSU 406, software 404, and microphone and speaker 408.

DPSU 406 may be, for example, without limitation, a computer, a cell phone, a tablet, a single chip, a combination of chips (e.g. a Raspberry Pi), a microcontroller, etc.

Software 404 can be stored on DPSU 406. Software 404 may be a set of instructions that may be used to send control signals to refracting device 410 and communicate between interface system 402, refracting device 410, and patient 412.

Microphone and speaker 408 may be one set of devices that may be used to output instructions and receive input to and from patient 412.

DPSU 406, alternatively, may be integrated into refracting device 410. Microphone and speaker 408 may also be integrated into DPSU 406. Microphone and speaker 408 may also be integrated into refracting device 410. Software 404 communicates with patient 412 and refracting device 410 through DPSU 406. Software 404 may be used to modify, for example, without limitation, values for spherical lens power, cylinder lens power, prism value and values of other mechanical components of refracting device 410. (e.g. shutter, which controls the opening and closing of the aperture of refracting device 410). Software 404 may also control microphone and speaker 408 (i.e. accepting input via the microphone, broadcasting messages via the speaker, etc.). When patient 412 speaks, the microphone captures the audio signal of patient 412. If refracting device 410 includes a monitor, software 404 may control the monitor. The monitor may be used to show, for example, without limitation, letters, patterns, images, words, videos, etc. to the patient. In such a case, software 404 communicates with refracting device 410, and thus controls the monitor. In another embodiment, software 404 does not control the monitor. In such a case, software 404 uses DPSU 406 to control the monitor. As will be appreciated by one skilled in the art, interface system may also include alternative input and output devices such as, without limitation, a mouse, a keyboard, a joystick, buttons etc. For example, patient 412 can communicate with software 404 via a joystick instead of speaking.

As shown in FIGS. 4B-4C, alternative input and output devices may be used to communicate with patient 412, such as speaker and camera 414 and monitor and camera 416.

With reference to FIG. 4B, for example, in system 401, a camera may be used to allow patient 412 to convey his/her choices via hand motion instead of speaking. The camera captures hand motions of patient 412 and sends the images and/or videos to software 404. Software 404 may interpret the images and/or videos and act accordingly. For example, without limitation, when software 404 may be used to ask patient 412 “Which views do you like? View 1 or view 2?”. Patient 412 can show two fingers to the camera to indicate a preference for view 2. The camera captures patient 412 showing two fingers and software 404 interprets that patient 412 prefers view 2.

With reference to FIG. 4C, for example, in system 401, a camera is used when the patient decides to convey choices via hand motion instead of speaking. The monitor is used to show the patient instruction from software 404. For example, the software can use the monitor to show the following message: “Which views do you like? View 1 or view 2?” As such, if patient 412 has difficulty hearing, patient 412 may still communicate with software 404. The monitor can also be used to display typical letters, words, symbols, etc. used during a refraction examination. In another embodiment, multiple monitors may be used, wherein a first monitor is used to show letters, words, symbols, etc. during a refraction examination and a second monitor is used to show patient 412 instructions from software 404.

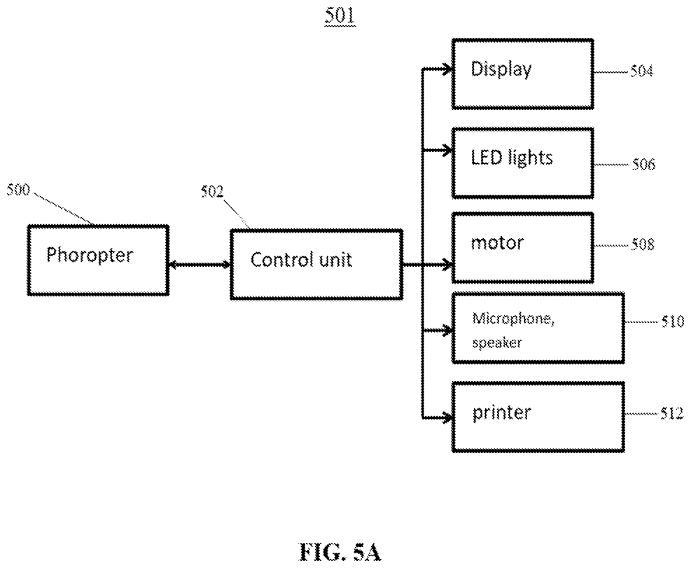

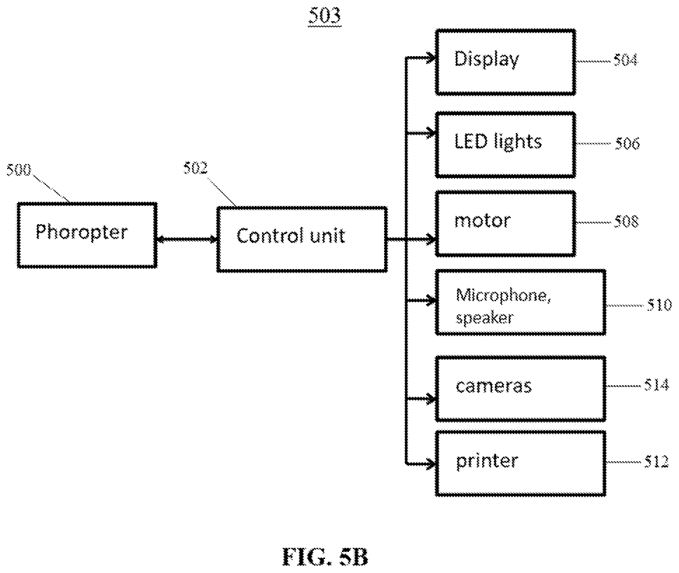

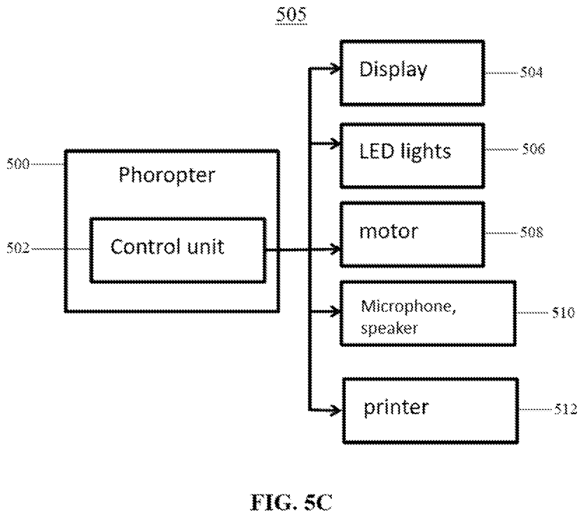

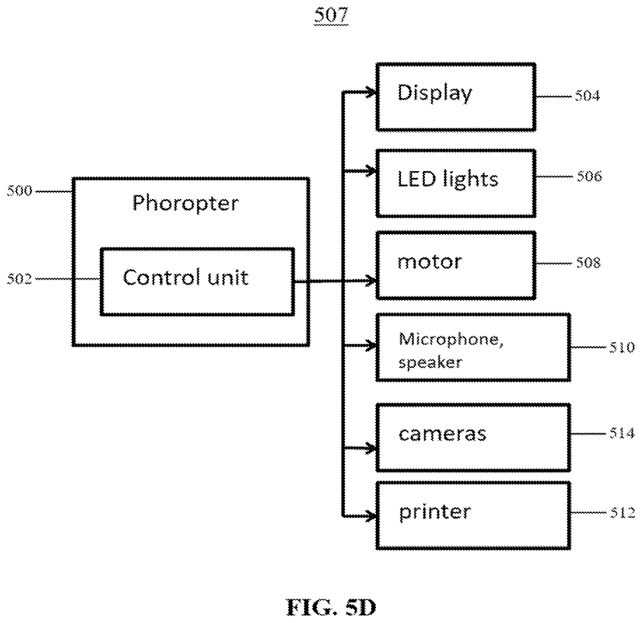

FIGS. 5A-5D illustrate exemplary automated refraction systems, wherein FIG. 5A shows a first system, FIG. 5B shows a second system, FIG. 5C shows a third system, and FIG. 5D shows a fourth system, in accordance with an embodiment of the present disclosure.

With reference to FIG. 5A, system 501 includes phoropter 500, control unit 502, display 504, LED lights 506, motor 508, microphone and speaker 510, and printer 512.

Phoropter 500 may include electrical units such as, without limitation, a power supply, semiconductor chips, semiconductor chipsets etc. The electrical units inside the phoropter 500 control various optical and mechanical components inside phoropter 500. Phoropter 500 may communicate with other hardware such as, without limitation, a tablet, cell phone, computers etc. Software may be installed and stored inside control unit 502. The software is executed by control unit 502.

Control unit 502 may be, for example, without limitation, a computer, a tablet, a single semiconductor chip, a semiconductor device, etc.

Display 504 may be any type of display, such as, without limitation, a monitor, a computer, a mobile phone, a tablet, etc.

LED lights may be used within system 501 to illuminate different components of the phoropter, as well as indicate the status of the automated refraction process. For example, without limitation, an LED light may be lit upon successful or unsuccessful termination of the automated refraction process. Additionally, LED light may also be a fluorescent light, an incandescent light, etc.

Motor 508 may be used to control aspects of phoropter 500. For example, without limitation, motor 508 may be used to control a reading rod coupled to phoropter 500. Upon actuation of motor 508, the reading rod may be placed into an active position from an inactive position, or may be placed into an inactive position from an active position. The active position may be where the reading rod is parallel with the ground and configured to display a reading card or display in front of a patient. The reading rod may be placed in the active position during, for example, a near vision test. The inactive position may be where the reading rod is perpendicular with the ground. The reading rod may be placed in the inactive position upon completion of the near vision test, or when not in use.

Control unit 502 controls various hardware, communicates with various hardware, performs software execution, governs input and output operations, etc. Control unit 502 communicates with phoropter 500. Control unit 502 may control phoropter 500. Control unit 502 also communicates with display 504, LED lights 506, motor 508, microphone and speaker 510, printer 512, etc. A reading rod may be coupled with a card holder configured to hold a near point reading card. The reading rod also may also be coupled with a motor. LED lights 506 may illuminate the near point reading card. LED lights 508 may also be positioned at the doctor's office. LED lights 508 may be positioned outside the door of the examination room. LED lights 508 may be positioned at multiple positions. LED lights 508 may illuminate the pupil of the patient through the eye apertures of phoropter 500.

With reference to FIG. 5B, in system 504, cameras 514 may be incorporated into the system. One camera may be mated to the reading rod. Another camera may be positioned in front of the patient to capture the patient's hand motions. Cameras 514 may be positioned at multiple positions throughout system 503.

With reference to FIGS. 5C-5D, control unit 502 may be integrated within phoropter 500, as in system 505 and system 507.

To initialize the refraction process, the patient is welcomed by an assistant and directed to sit in a chair of an examination room. An introduction video is shown to the patient about the refraction process. The introduction video may also be available beforehand such that the patient has access to the introduction video before or after the refraction examination.

The assistant may input information of the patient into a user interface, including, for example, without limitation, the patient's age, visual acuity etc.

The assistant may adjust the pupil distance via software such that each eye of the patient is aligned with the center of the left eye aperture and right eye aperture of the phoropter, and an automated refraction process may be initiated. The assistant may adjust a level attached to a front or side surface of the phoropter to ensure that the phoropter is leveled. The pupil distance may also be adjusted by the patient, or automatically adjusted by the system.

“Assistant” as used herein is the assistant in the clinic who does not have a doctor degree.

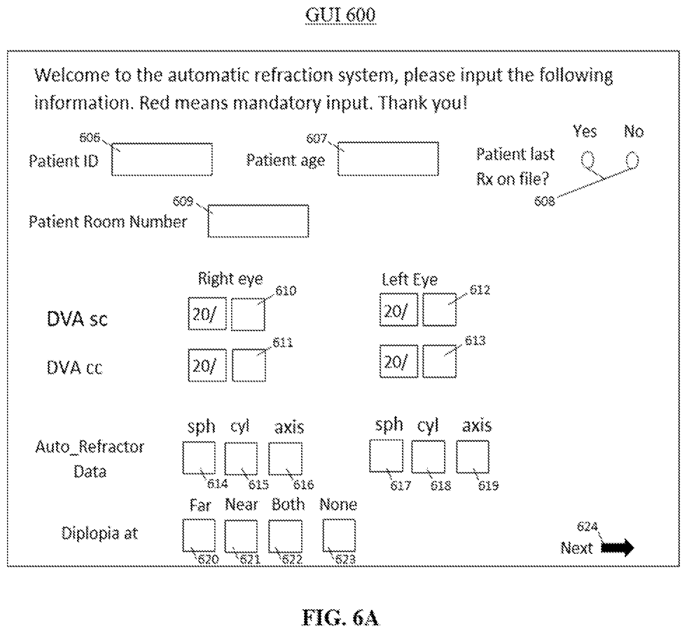

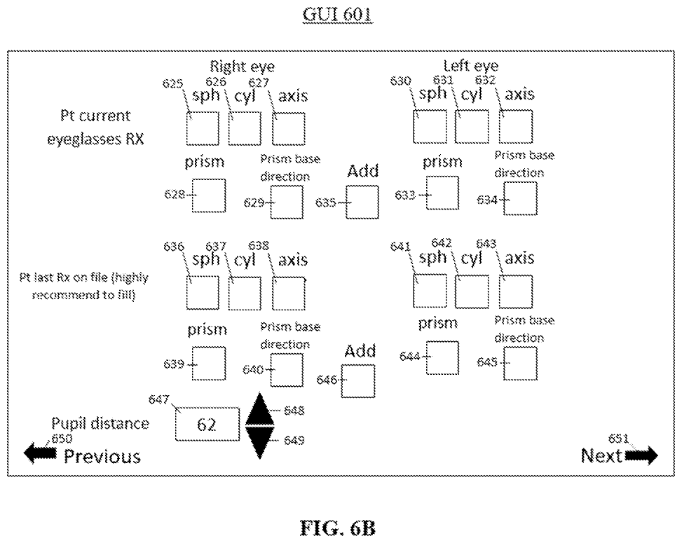

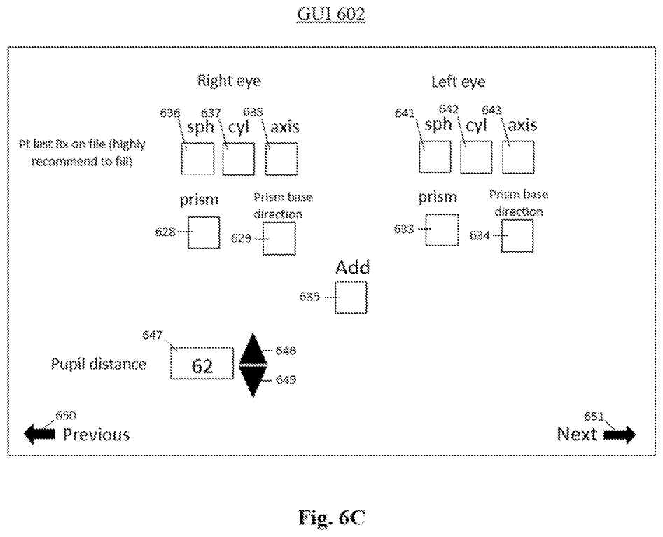

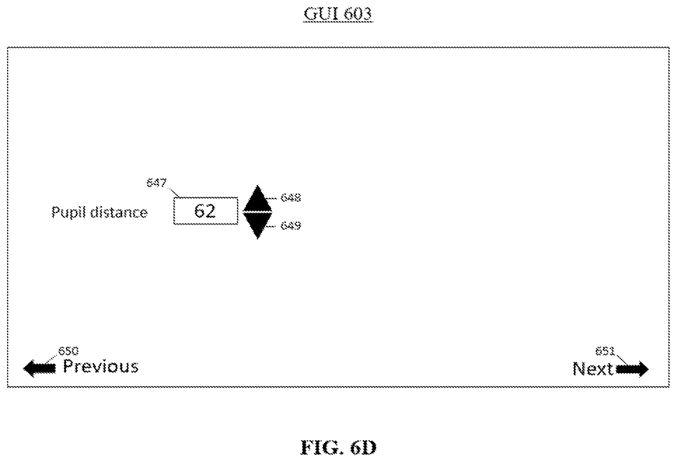

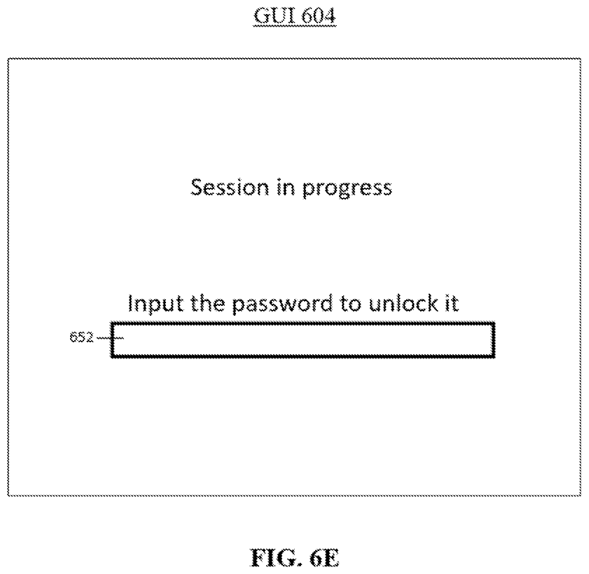

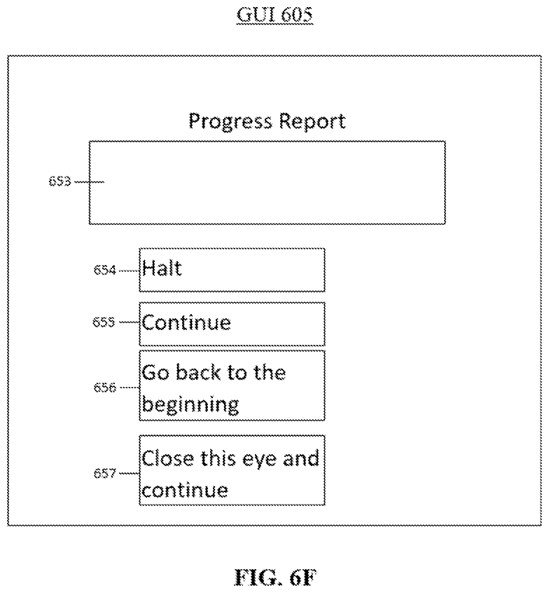

FIGS. 6A-6F illustrate exemplary user interfaces of an automated refraction system, wherein FIG. 6A shows a first interface, FIG. 6B shows a second interface, FIG. 6C shows a third interface, FIG. 6D shows a fourth interface, FIG. 6E shows a fifth interface, and FIG. 6F shows a sixth interface, in accordance with an embodiment of the present disclosure.

The patient or assistant may input information of the patient including, for example, without limitation, the patient's age, visual acuity etc. as shown in FIGS. 6A-6D.

As shown in FIG. 6A, graphical user interface (GUI) 600 may be used to obtain initial patient information. Required information in GUI 600 may include, for example, Patient ID 606, Patient age 607, Patient last Rx on file 608, Patient room number 609, right eye DVA sc 610, and left eye DVA sc 612, wherein DVA sc may be the patient's vision without corrected vision. Additional information may include, for example, Auto_Refractor Data including right sph 614, right cyl 615, right axis 616, left sph 617, left cyl 618, left axis 619 and diplopia data including far 620, near 621, both 622, and none 623. It should be noted that only one box for the diplopia data may be filled. For example, if the patient has diplopia at a far distance, far 620 should be filled.

After information is input to GUI 600 and next button 624 is selected, the patient input module may continue to GUI 601 as shown in FIG. 6B, GUI 602 as shown in FIG. 6C, or GUI 603 as shown in FIG. 6D, depending on what type of information is still required from the patient.

As shown in FIG. 6B, GUI 600 may be used to obtain supplementary patient information. Required information may include, for example, Pupil distance 647. Additional information may include the patient's current eyeglass prescription, including right sph 625, right cyl 626, right axis 627, right prism 628, right prism base direction 629, left sph 630, left cyl 631, left axis 632, left prism 633, left prism base direction 634, and add value 635, and patient's last prescription on file including right sph 636, right cyl 637, right axis 638, right prism 639, right prism base direction 640, left sph 641, left cyl 642, left axis 643, left prism 644, left prism base direction 645, and add value 646. Further, pupil distance may be controlled using up arrow 648 and down arrow 649. Previous button 650 may be used to go back to GUI 600, while next button 651 may be used to proceed.

As shown in FIG. 6C, GUI 602 may include the same information as GUI 601 except for the patient's current eyeglass prescription.

As shown in FIG. 6D, GUI 603 may include the same information as GUI 602 except for the patient's last prescription on file.

As shown in FIG. 6E, GUI 604 may be used to log into an automated refraction process interface via password field 652.

As shown in FIG. 6F, GUI 605 may include additional commands and information during or after an automated refraction examination. For example, without limitation, GUI 605 may include progress report 653, halt 654, continue 655, go back to the beginning 656, and close this eye and continue 657.

The required information may be mandatory information and may be input by the assistant. The assistant may enter patient information into the input page shown in FIG. 6A first. The assistant and doctor may then choose between the input pages shown in FIGS. 6B-6D. The input page shown in FIG. 6B may provide more information, while the input page shown in FIG. 6D may be faster.

The patient may also adjust the pupil distance via software to ensure each eye of the patient can see through the center of the left eye aperture and right eye aperture of the phoropter and then start the refraction process controlled by the software. The patient may adjust the bubble level attached to the front and/or the side surface of the phoropter to make sure the phoropter is leveled. The patient may also input the information detailed in FIGS. 6A-6D.

Patient room number refers to the number of the room where patient is to conduct the refraction examination in the clinic. “DVA” refers to distance visual acuity. “sc” refers to the patient's uncorrected vision. “cc” refers to the patient's corrected vision. The assistant can directly input patient information via one or more input devices, such as, without limitation, a keyboard, voice recognition module, etc. The assistant may also use a scroll down menu including, for example, without limitation, the following numbers: 10, 15, 20, 20-, 25, 25-, 30, 30-, 40, 50, 60, 70, 80, 100, 150, 200, 400, 800, 100, CFF @ 3 feet, CFF @ 6 feet, light perception (LP), no light perception (NLP) to input visual acuity information. The assistant may also use a scroll down menu to input “Auto_refractor data” and input the diplopia status. Patient information may also include, for example, without limitation, near visual acuity (NVA) cc, and/or NVA sc. The unit of prism is prism diopter (pd). The prism may be, for example, without limitation, 0.25, 0.5, 0.75, 1, 2 3, 4, 5, 6, 8, 10. The direction angle may be between 0 and 179 degrees steps of 1 degree.

“Sph” refers to the spherical value of the refractive error. Sph can have a range of [+26.75, −29] diopter with 0.25 steps. “Cyl” refers to the cylinder value of the refractive error with a range of [0, −8.75] diopter with 0.25 steps. Axis refers to the axis angle of the cylinder lens with a range is [0, 179] degree with 1 degree steps. Patient ID may be assigned to each patient by the clinic. The values shown in FIGS. 6A-6D may be retrieved from the patient's existing record of an electrical health record (EHR) system. When the software calculates the angle, the value of the angle may be less than zero or more than 179 deg. The software may add 180 or subtract 180 to the angle so that the angle can fall into [0, 179] range. The angle refers to axis and prism base direction.

When the assistant selects the “next” button” of FIG. 6A, the software may check if the required information has been entered. If the required information is properly entered, the software may proceed to the interface shown in FIG. 6B, the interface shown in FIG. 6C, or the interface shown in FIG. 6D. If the required information is improperly or incompletely entered, a warning window may be shown instructing the assistant to properly input the required information. When the assistant clicks the “previous” button in FIG. 6B, FIG. 6C, or FIG. 6D, the software may go back to the interface shown in FIG. 6A.

The patient information or a portion thereof and the data collected during the refraction examination, including video and or audio files, may be stored in a local or a cloud based drive. The stored information may be encrypted. The information may be stored on one or more databases.

“Patient last Rx on file” refers to the patient's last vision eyeglass prescription data. The patient vision eye glass prescription data may be automatically input from the EHR if available.

“Pt” or “pt” refers to the patient. “Eye glasses Rx” refers to eyeglass prescription. The assistant may manually input the pupil distance data. The assistant may also use triangle buttons or input means to adjust the pupil distance with a step of 0.5 mm. The software may send the input data to the phoropter and the phoropter may adjust the distance between the left and the right eye apertures accordingly. “Rx” mean prescription. “Vision Rx” refers to refractive error prescription. When the assistant clicks the “next” button in FIGS. 6B-6D, the software may proceed to the refraction process. As used herein: (a) Rx 1 refers to Auto_refractor data; (b) Rx 2 refers to patient current eyeglass prescription; (c) Rx 3 refers to patient last prescription on file. The software may check if there is at least one set of data available for each eye among Rx 1, Rx 2, and Rx 3. If none of the data is available, the software returns to the interface shown in FIG. 6A and instructs the assistant to input additional information.

The database may allow the assistant and the doctor (a) to find one specific patient based on a patient ID; (b) to edit the existing patient file and change data within a fixed amount of time (e.g. within 24 hours); (c) to add new sessions for an existing patient; (d) to display the list of sessions of the same patient based on the date and show relevant data of a specific session; (e) to delete a session if it is within a certain amount of time (e.g. within 24 hours) post examination. The database may lock patient data after a certain amount of time (e.g. 24 hours) post examination to avoid any more editing. Once the software is running, patient data may be locked and password protected. The software may lock the screen after, for example, 30 seconds if there is no input detected, as shown in FIG. 2E. When the assistant unlocks the screen by inputting a proper password, the software may display the window shown in FIG. 6F showing the current status of the refraction (e.g. “axis testing in the right eye”, “sph testing in the left eye”, “binocular balance”, “near vision”, etc.). Additional options may also be available, such as, without limitation, “Halt”, “Go back to the beginning”, “Close this eye and continue”, “Continue”, etc. If the assistant selects “Halt”, the software may display “are you sure? Yes or no” via a pop-up screen or speaker. If the assistant selects “yes”, the software may stop the operation of the phoropter and the phoropter may be placed in a waiting mode. If the assistant selects “Continue”, the software continues the refraction examination.

FIG. 7 illustrates a flowchart showing an automated refraction process, in accordance with an embodiment of the present disclosure. Automated refraction process 700 may begin with a step S702, wherein patient information is input via a patient information input module as shown in FIGS. 6A-6F. Data related to patient vision prescription may include auto refractor data, patient last prescription on file, and patient current eyeglasses prescription. In a step S704, the data related to patient vision prescription may be reconciled via a data reconciliation module. In a step S706, the output of the data reconciliation module may be confirmed to be within possible prescription ranges via a sanity check module. In a step S708, the patient's vision performance at 20/60 is measured via a 20/60 line clear module. In a step S710, the patient's normal speaking speed is measured via a speaking speed measurement module. In a step S712, the patient's vision performance at 20/40 is measured via a 20/40 line clear module. In a step S714, the patient's cyl value is determined to be equal to 0 or not. If yes, in a step S716, it may be determined if the patient needs a cylinder lens prescription via a cyl search module. In a step S718, the patient's cyl value may be again compared to 0. If cyl is not 0 as determined in either of step S714 or S718, the axis angle of the cylinder lens may be refined via an axis module. In a step S722, the proper cyl value of the patient may be found via a cyl module. In a step S724, the axis of the prior prescription and the newly found axis may be compared via an axis compare module. After S724 or if yes in S718, the cyl of the prior prescription and the newly found cyl may be compared via a cyl compare module.

In a step S728, it may be determined whether the display shows 20/40 size letters/words or 20/60 size letters/words. If no, in a step S732, the patient's vision may be optimized at 20/60 via an optimization at 20/60 module. If the patient sees clearly at 20/40, automated refraction process 700 may continue to a step S730, wherein the patient's vision may be optimized at 20/40 via an optimization at 20/40 module. Depending on the results from S730 and S732, automated refraction process 700 may continue with a step S736 or a step S734, wherein the patient's prescription may be further refined via a read 20/20 size module or a read large size model, respectively. In a step S738, if the patient's other eye has not been tested yet, automated refraction process 700 may be repeated between steps S702-S736 for the patient's other eye. In a step S740, the patient's sph value for each individual eye may be adjusted via a binocular balance module. In a step S742, the patient's near vision may be tested via a near vision test module. In a step S744, the patient's prism value may be tested in a prism test module.

The software may define minsph=sph value from the output from the “data reconciliation” module−1.00 and mincyl=cyl value from the output from the “data reconciliation” module−1.00. In any module, once the sph or cyl value in the phoropter is less than minsph, or mincyl value, the software may not further reduce the value of sph or cyl in this module.

Upon successful termination of automated refraction process 700, the results of automated refraction process 700 and a completion report may be sent to a database. In another embodiment, the completion report may include the results. The completion report may also include an alert for notifying the appropriate staff that automated refraction process 700 is complete. The alert may be, for example, without limitation, an audio message broadcasted via a speaker, a notification light outside the examination room, etc.

FIG. 8 illustrates a flowchart showing an exemplary data reconciliation module, in accordance with an embodiment of the present disclosure. The output from data reconciliation module 800 serves as the starting point of the refraction. As will be appreciated by one skilled in the art, alternative methodologies may be used to determine a starting point for the refraction examination. In a step S802, it may be determined whether the patient's corrected or uncorrected vision is better than 20/30. In the present embodiment, Rx1 refers to auto refractor data, Rx2 refers to the current eyeglass prescription, and Rx3 refers to the last prescription on file. If yes, in a step S806, the most recent between Rx2 and Rx3 is chosen as a starting point if available. If unavailable, Rx1 is chosen. If no, in a step S804, Rx1 is chosen if available. If unavailable, the most recent between Rx2 and Rx3 is chosen. The output from data reconciliation module 800 specifies the spherical lens power, cylinder lens power, axis angle, prism power and/or angle for each eye. The phoropter takes the output from data reconciliation module 800 and positions the spherical lens with a corresponding power and name “sph”, cylinder lens with a corresponding power and name “cyl” and angle with the name “axis”, and/or prism with a corresponding power and angle in front of the patient's eyes.

The software may send ASCII code or other forms of coding signal to a USB port or other type of ports of the phoropter; the phoropter then performs hardware operations on its mechanical components such as closing or opening the eye aperture etc. As will be appreciated by one skilled in the art, various control schemes for the phoropter may be used in the present embodiment, including, for example, without limitation, wireless communication protocols, wired communication, etc.

The software has a list of messages/audio files saved in a database and broadcasts the messages through a speaker during the refraction examination to instruct the patient and illustrate the current progress of the refraction examination. For example, without limitation, the software can direct the speaker to broadcast: “Can you see this line of letters clearly? Please say: yes, no, or a little blurry”. The messages may also be displayed on a screen such that hearing impaired patients can read the messages.

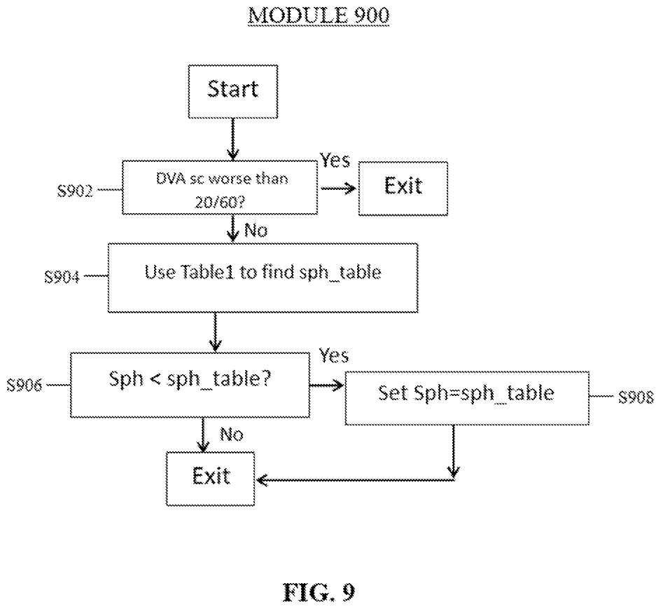

FIG. 9 illustrates a flowchart showing an exemplary sanity check module, in accordance with an embodiment of the present disclosure. Sanity check module 900 and table 1 may ensure that the data output from data reconciliation module 800 does not deviate too far from possible visual prescription values. In a step S902, the patient's vision is compared to a 20/60 threshold. If the patient's vision is worse than 20/60, professional intervention may be required, and the process exits. If better than 20/60, in a step S904, an sph_table value may be determined based the patient's DVA sc and table 1. If the patient's initial sph value is less than the sph_table value as determined in a step S906, the patient's sph value will be set to the sph_table value in a step S908. If not, the automated refraction examination may continue with the patient's initial sph value. For example, if an individual can see 20/25 size letters without wearing any vision correction devices such as glasses and the output data from data reconciliation module 800 shows the spherical error is −2.00D, it may not pass the sanity check because it is extremely unlikely for someone who can read 20/25 letters without eyeglasses to require −2.00 diopter sph glasses. Sanity check module 900 then uses the value from table 1, which is −0.75 diopter, to be the starting point of the refraction.

TABLE 1

| DVAsc | sph_table |
|---|---|
| 20/20 or 20/20− | −0.50 |
| 20/25 or 20/25− | −0.75 |
| 20/30 or 20/30− | −1.00 |
| 20/40 | −1.25 |
| 20/50 | −1.75 |
| 20/60 | −2.00 |

Voice recognition modules in the present disclosure generally return a text document as a response to an input audio file. The voice recognition module may be stored and executed in the control unit. Such an arrangement may be referred to as a local voice recognition module. The patient's audio files are fed into the local voice recognition module, which process the audio files and returns a text file. In the present embodiment, voice recognition modules may also be stored and operated remotely. Such an arrangement may be referred to as a cloud based voice recognition module. The patient's audio files are fed into the cloud based voice recognition module, which process the audio files and returns a text file. A hybrid type voice recognition module may also be used. In the hybrid type voice recognition module, both local voice recognition module stored and executed on a local control unit and cloud based voice recognition module are used in combination. When the patient speaks into the microphone, the patient's audio file is saved onto a local hard disk drive and/or a cloud drive. The audio file may be fed into both the cloud based voice recognition module and the local voice recognition module. Since the patient is presented with a limited selection of answers, the response from the patient is likely to be from a small set of possible answers. If these answers are short answers such as “yes”, “no”, “blurry” etc., the patient may be given a short time period in which to reply. For example, without limitation, the time limit may be 10 seconds, as the patient is expected to finish responding in 10 seconds. As will be appreciated by one skilled in the art, the 10 second time period is adjustable, and may vary between different types of patients. If the response is expected to be longer, such as when the patient is to respond with multiple words, the time period for reply may be, for example, 40 seconds. The voice recognition modules, as will be described below with reference to FIGS. 10-11, are setup to recognize multiple languages such as, but not limited to, English, Chinese, Spanish, French, etc. If the local voice recognition module has a null return or the return does not fall into the expected answer set, the response from the cloud based voice recognition module may be used. If the return from the cloud based voice recognition module falls into the expected answer set, the response from the cloud based voice recognition module may be used. If both voice recognition modules do not generate a useful response, the question may be repeated to the patient multiple times. In the present embodiment, the question may be repeated 3 times. However, the question may be repeated for a greater or lesser number of times, depending on a specific situation. An alternative hybrid voice recognition module may input the audio file into the local voice recognition module first. If the local voice recognition module does not output a meaningful response, the audio file may be input into the cloud based voice recognition module. If both voice recognition modules do not output a useful response, the question process may be repeated multiple times. In the present embodiment, the question process may be repeated 3 times. Alternatively, the hybrid voice recognition module may input the audio file into the cloud based voice recognition module first. If the cloud based voice recognition module does not output a meaningful response, the audio file may be input into the local voice recognition module. If both voice recognition modules do not output a useful response, the question process may be repeated for the patient multiple times. In the present embodiment, the question process may be repeated 3 times. In the hybrid voice recognition module, the correction rate from both local and cloud based voice recognition modules may be compared to further improve the voice recognition modules. One index that may be used is corr_index=100%, the correction rate from the local voice recognition module or the correction rate from the cloud based voice recognition module. The patient's audio files and the return from the voice recognition modules along with other patient information may be encrypted and securely saved into a local and/or cloud based database. The microphone may be a wireless microphone, or directly connected to the control unit via a cable. The microphone may also be integrated into the phoropter.

FIG. 10 illustrates a flowchart showing an exemplary first voice integration module, in accordance with an embodiment of the present disclosure. The automated refraction examination includes a series of conversations with the patient. The software presents the patient with two views by changing certain optical and/or mechanical components in the phoropter and asks the patient to compare the two views. In a step S1002, a message may be broadcasted with a 5 second waiting period. For example, while the display shows a line of letters of 20/60 size, the software broadcasts a message: “We will show you two views, please tell me which view is clearer. This is the 1st view”. The software may then wait 5 seconds for the patient to fully appreciate the image quality of the first view. In a step S1004, hardware changes of the phoropter may be conducted and a second message may be broadcasted with a 5 second waiting period. For example, the software changes the value of the spherical lens and broadcasts the second message “This is the 2nd view”. At the end of the broadcast, the patient may be asked to choose between the two views.

In a step S1006, while waiting for a response from the patient, a text file returned by the voice recognition software may be checked every 2 seconds. For each conversation, a list of key words may be used corresponding to possible answers. For example, the key words may include “image 1”, “image 2”, “same” and “repeat”. If the patient says repeat, the software may repeat showing the patient the two views for multiple times. By comparing the patient's answer to the key words, the software can make decisions and proceed through the automated refraction examination. In a step S1008, it may be determined whether or not the patient has responded in a 10 second window. If no, in a step S1010, the number of times a response was not recorded is determined. If beyond a certain threshold (in the present embodiment, 3 times) first voice recognition module 1000 may exit with an error. If not, in a step S1018, the hardware settings of the phoropter may be reset, and first voice recognition module 1000 may be repeated. For short answers such as “yes” and “no”, the voice recognition module may be turned off 10 seconds. The 10 second window may be adjustable depending on factors such as the type of response, the age of the patient, etc. For longer answers, the voice recognition module may use a 40 second window for response. In a step S1014, if the patient responded within the time window, it may be determined whether or not the patient's response contained any key words. If yes, first voice recognition module 1000 exits successfully. If no, in a step S1016, the number of times a key word is not found is determined. If beyond a certain threshold (in the present embodiment, 3 times), first voice recognition module 1000 may exit with an error. If not, in a step S1018, the hardware settings of the phoropter may be reset, and first voice recognition module 1000 may be repeated.

FIG. 11 illustrates a flowchart showing an exemplary second voice integration module, in accordance with an embodiment of the present disclosure. Second voice integration module 1100 may ask the patient to evaluate one view and indicate his/her decision. In a step S1102, a message may be broadcasted with a 5 second waiting period. For example, the software shows one line of 20/25 size letters or words on the display and broadcasts the following message “Can you see this line clearly? You can say yes, no, blurry, or repeat. You can begin now”. In a step S1104, the software then checks the return from the voice recognition module every 2 seconds. If the patient says “repeat”, the software may repeat showing the patient two view for a few times. In another scenario, the software asks the patient to read out the letters or words shown on the display and then the software determines the correction rate of the patient's reply. In a step S1106, it may be determined whether or not the patient has responded in a 10 second window. If no, in a step S1108, the number of times a response was not recorded is determined. If beyond a certain threshold (in the present embodiment, 3 times) Second voice integration module 1100 may exit with an error. If not, Second voice integration module 1100 may be repeated starting from S1102. For short answers such as “yes” and “no”, the voice recognition module may be turned off 10 seconds. The 10 second window may be adjustable depending on factors such as the type of response, the age of the patient, etc. For longer answers, the voice recognition module may use a 40 second window for response. In a step S1112, if the patient responded within the time window, it may be determined whether or not the patient's response contained any key words. If yes, second voice integration module 1100 exits successfully. If no, in a step S1114, the number of times a key word is not found is determined. If beyond a certain threshold (in the present embodiment, 3 times), in a step S1110, second voice integration module 1100 may exit with an error. If not, second voice integration module 1100 may be repeated from step S1102.

If first voice recognition module 1000 or second voice recognition module 1100 exit unsuccessfully (i.e. S1012 and S1110, respectively), an error report may be sent to a database to alert, for example, without limitation, the optometrist, the doctor, the assistant, the technician, etc. such that appropriate action may be taken. Similarly, if the patient is using an alternative input means other than voice recognition and fails to respond multiple times, an error report may be sent to the database. For example, without limitation, a patient may lose consciousness (e.g. from low blood sugar) and is unable to respond to prompts from first voice recognition module 1000 or second voice recognition module 1100, an alert may be sent out to the relevant staff. In one embodiment, additional alert notifications may be sent, such as, without limitation, an audio message may be broadcasted over a speaker, an alert light outside the examination room may be lit, a message may be sent to the appropriate staff, etc.

First voice recognition module 1000 and second voice recognition module 1100 may ask the patient to compare two different views. The software may name the first view as image 1 and the 2nd view as image 2 and ask the patient “Which view do you prefer? Image 1 or image 2”. The software may also name the first view as image 3 and the 2nd view as image 4 and ask the patient “which view do you prefer? Image 3 or image 4”. By giving the views different names during the automated refraction examination, the names themselves may have less of an impact on a patient's choice between different views, especially for patient's having a strong bias or preference for certain numbers.

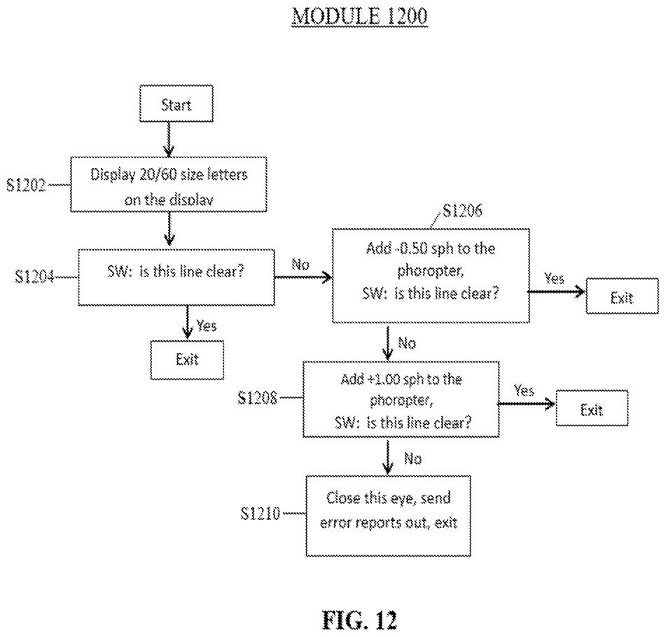

FIG. 12 illustrates a flowchart showing an exemplary 20/60 line clear module, in accordance with an embodiment of the present disclosure. In a step S1202, 20/60 size letters may be displayed to the patient. In a step S12004, the software (SW) may broadcast the message: “Is this line clear?” and use either first voice recognition module 1000 of FIG. 10 or second voice recognition module 1100 of FIG. 11 to process results from the patient. In the present embodiment, the keywords “yes”, “no”, and “repeat” are expected from the patient. Optionally, the software may broadcast a message indicating the expected responses: “You can say yes, no, or repeat”. If the patient responds “yes”, the automated refraction process may proceed to the next module. If the patient responds “no”, in a step S1206, the sph of the phoropter may be adjusted by −0.50 and the message “Is this line clear?” may be repeated to the patient. If yes, 20/60 line clear module 1200 exits and the automated refraction process continues to the next module. If no, in a step S1208, the sph of the phoropter may be adjusted by +1.00 and the message “Is this line clear?” may be repeated to the client. If yes, 20/60 line clear module 1200 exits and the automated refraction process continues to the next module. If no, in a step S1210, 20/60 line clear module 1200 may exit with an error. For example, if initially the spherical lens inside the phoropter in front of the patient's right eye has −2.50 diopter power and the patient responds “no”, the voice recognition process returns “no” to the software. Then the software calculates −0.50+(−2.50)=−3.00. The −2.50 diopter spherical lens may then be replaced with a −3.00 diopter spherical lens (inside the phoropter) in front of the patient's right eye. Instead of a line of letters, the software may also display words or figures on the display.

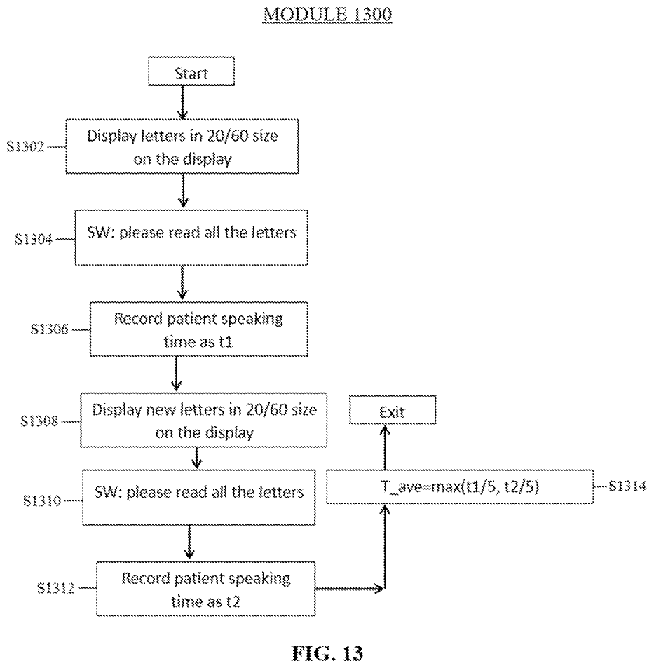

FIG. 13 illustrates a flowchart showing an exemplary speaking speed measurement module, in accordance with an embodiment of the present disclosure. Speaking speed measurement module 1300 may be used to measure the speech speed of the patient to determine when the patient speaks at a normal pace. In a step S1302, 20/60 size letters may be displayed to the patient. In a step S1304, a message may be broadcasted to the patient instructing them to read the letters on the display. In a step S1306, a first speaking time t1 may be recorded. In a step S1308, a new set of 20/60 size letters may be displayed to the patient. In a step S1310, the patient may be instructed to read the new set of letters. In a step S1312, a second speaking time t2 may be recorded. In a step S1314, the average speaking time T_ave may be calculated according to T_ave=max(t1/5, t2/5), wherein 5 corresponds to one less than the number of letters in the letter sets (i.e. in the present embodiment, 6 letters were shown). Instead of letters, words may be presented to the patient and the patient may be asked to read the words to measure the patient's speaking speed. Alternative formulas may be utilized to calculate T_ave, such as, but not limited to, T_ave=(t1/5+t2/5)/2, etc. The threshold of the speaking speed may be set at 1, and the threshold may be relaxed or tightened depending on the patient. If T_ave is less than 1, T_ave may be set to be 1. Speaking speed may be used as an indicator of confidence in the patient's responses. For example, without limitation, if the patient's normal speaking speed is 1.5 seconds per word and the patient slows down to 3 seconds per word when reading words at 20/20 size, the patient may have difficulty in seeing the words at that distance, which may prompt the software to further refine the patient's prescription by adjusting the spherical lens, cylinder lens, and axis angle for the cylinder lens. In general the “SW:” format indicates that the voice recognition processes of FIG. 10 or FIG. 11 is used.

One means for broadcasting a message is inputting the message in text format into a module, translating the message into an audio format, and sending the audio message word by word to the speaker so that the speaker broadcasts the message. In another embodiment, a voice may be recorded and saved as audio files. When needed, the software may retrieve an audio file from the database and send the audio file to the speaker to broadcast the audio file accordingly.

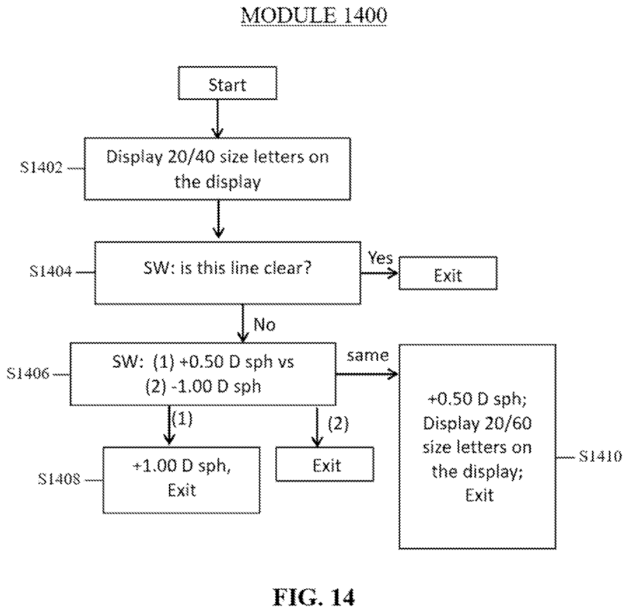

FIG. 14 illustrates a flowchart showing an exemplary 20/40 line clear module, in accordance with an embodiment of the present disclosure. In a step S1402, 20/40 size letters may be displayed to the patient. In a step S1404, the message “Is this line clear?” may be broadcasted to the patient using the voice recognition process shown in FIG. 11. If yes, the automated refraction process may continue to the next module. If no, two views will be presented to the patient: (1)+0.5 D sph and (2) −1.00 D, wherein the phoropter is adjusted to add +0.5 D or −1.00 D to the current spherical lens. Using the voice recognition process shown in FIG. 10, a response is recorded from the patient. If (1) is selected by the patient, in a step S1408, the phoropter may be adjusted to add +1.00 D sph and the automated refraction process may continue. If (2) is selected, no adjustment is needed and the automated refraction process may continue. If the patient indicates that the (1) and (2) are the same, the phoropter may be adjusted by adding +0.50 D sph, 20/60 size letters may be displayed to the user, and the automated refraction process may continue. For example: After S1404 and the patient responds “No”, 20/40 clear module 1400 may continue to S1406. In the present scenario, the spherical lens power in the phoropter is −3.00 D. The software first calculates: +0.50+(−3.00)=−2.50. The software then replaces the current spherical lens for the eye under testing with spherical lens with −2.50 D power. The software broadcasts the message “We will show you two views now. Please tell me which view is clearer. This is image 1”. The software then waits for 5 seconds. The software calculates: −1.00+(−2.50)=−3.50. The software then replaces the current spherical lens for the eye under testing with a spherical lens with −3.50 D power. The software broadcasts the message “This is image 2. Please tell me which image is clearer. You can say image 1, or image 2, same, or repeat. You can start now.” The software then checks the return from the voice recognition module and continues accordingly. “+1.00D sph” means the software sends a command to the phoropter to replace the current spherical lens in front of the patient eye with another spherical lens with a power +1.00 D.

FIG. 15 illustrates a flowchart showing an exemplary cylinder (cyl) search module, in accordance with an embodiment of the present disclosure. When the cylinder lens is initially set to zero, the software uses cyl search module 1500 to determine if the patient needs a cylinder lens prescription. The software uses a JCC lens to determine the patient's cylinder error and a corresponding axis. The software positions the JCC at different angles. In a step S1502, the JCC may be initialized with J_axis set at 0 degrees. In a step S1504, the patient may be asked to compare (1) a view with the JCC positioned at the angle specified by J_axis and (2) a view with the JCC positioned at an angle of J_axis+90 deg. If the patient selects (1), in a step S1506, a cylinder lens is selected with −0.5 D power, a related axis angle is set at J_axis, +0.25 is added to the sph value, and the cylinder lens is positioned in front of the patient's eye. If the patient selects (2) or indicates that the views are the same, in a step S1508, the value of the J_axis is checked. If not equal to 135, in a step S1510, the J_axis value is increased by 45 degrees and cyl search module 1500 loops back to S1504. If J_axis is equal to 135, in a step S1512, the JCC is removed and cyl search module 1500 exits. Axis means the axis angle for the cylinder lens inside the phoropter. “(1), (2), same” are expected responses from the patient via the voice recognition module.

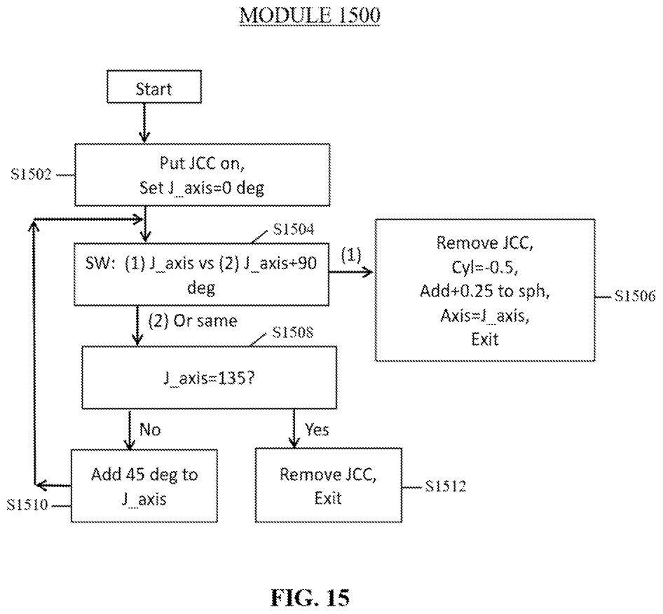

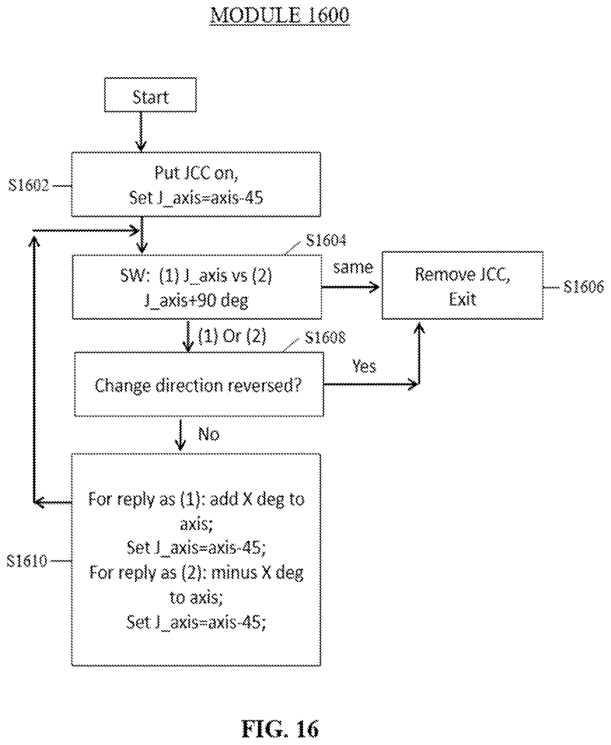

FIG. 16 illustrates a flowchart showing an exemplary axis module, in accordance with an embodiment of the present disclosure. Axis module 1600 may be used to refine the axis angle of the cylinder lens. “X degree” is specified in Table 2 below. The ANSI standard specifies tolerance of the cylinder lens axis angle. Thus Table 2 is based on the ANSI standard. In Table 2, the X value is set to be 1 degree more than the ANSI standard tolerance value. As will be appreciated by one skilled in the art, there are other ways to set the values in Table 2. As used in axis module 1600, the adjustment of the axis is referred to as “change”. When the axis is increased compared to its original value, its change direction is “+”. When the axis is decreased compared to its original value, its change direction is “−”. For example, the previous two times the axis gets adjusted, the change direction is “++”. If the next time the axis is adjusted the change direction is “−”, the software determines that the change direction reversed. Axis module 1600 may begin with a step S1602 wherein the JCC is initialized with J_axis set to 45 degrees less than the current axis. In a step S1604, two views may be presented to the patient: (1) JCC set at J_axis and (2) JCC set at J_axis+90 degrees. The message “We will show you two views here. Please tell us which view is clearer. You can say image 1, image 2, same, or repeat” may be broadcasted to the patient. If the change direction is not reversed, as determined in S1608, and the patient replies that they prefer “(1)”, the software adds X degree to the axis. If patient replies that they prefer “(2)”, the software subtracts X degree from the axis. Axis module 1600 may then loop back to S1604. If the change direction was reversed, as determined in S1608, axis module 1600 exits successfully in a step S1606. Similarly, if the patient indicates that (1) is the same as (2), axis module 1600 exits successfully in S1606. It shall be noted that the JCC lens is different from the cylinder lens.

TABLE 2

| Cyl Value | X |
|---|---|
| −0.25 | 15 |
| −0.50 | 8 |
| −0.75 | 6 |
| −1.00, −1.50 | 4 |
| <−1.50 | 3 |

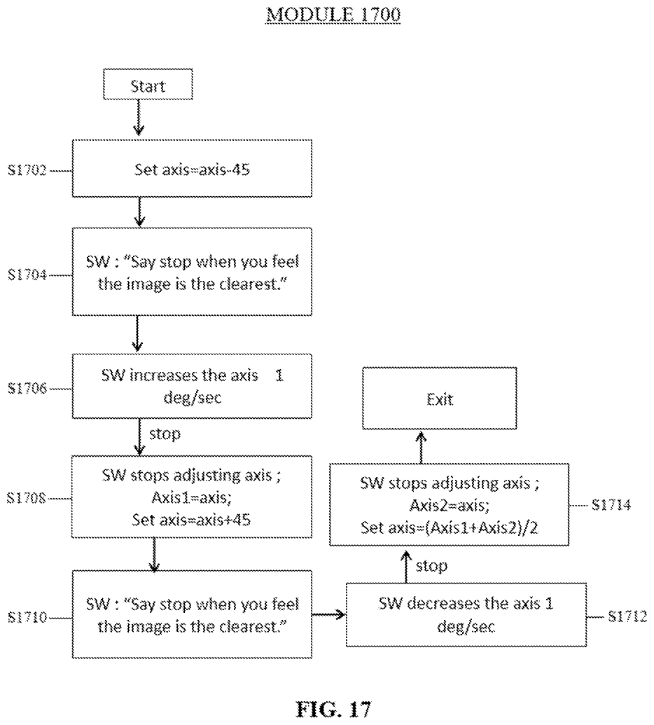

FIG. 17 illustrates a flowchart showing an exemplary alternative axis module, in accordance with an embodiment of the present disclosure. In alternative axis module 1700, a JCC lens is not utilized. Instead, the axis of the cylinder lens is gradually adjusted and the patient must indicate when the patient believes the image quality is the best. Alternative axis module 1700 may begin with a step S1702 wherein the axis is initialized to 45 degrees less than the current value. In a step S1704, first voice recognition module 1000 or second voice recognition module 1100 may be used to broadcast the message “Say stop when you feel the image is clearest” to the patient. In a step S1706, alternative axis module 1700 may increase the axis by 1 degree per second until stopped by the patient. In a step S1708, the current axis may be stored as Axis 1, and the axis angle may be increased by 45 degrees. In a step S1710, alternative axis module 1700 may again broadcast the message “Say stop when you feel the image is the clearest”. In a step S1712, the axis may be decreased by 1 degree per second until stopped by the patient. In a step S1714, the current axis may be stored as Axis2, and the axis angle may be set to the average between Axis 1 and Axis2.

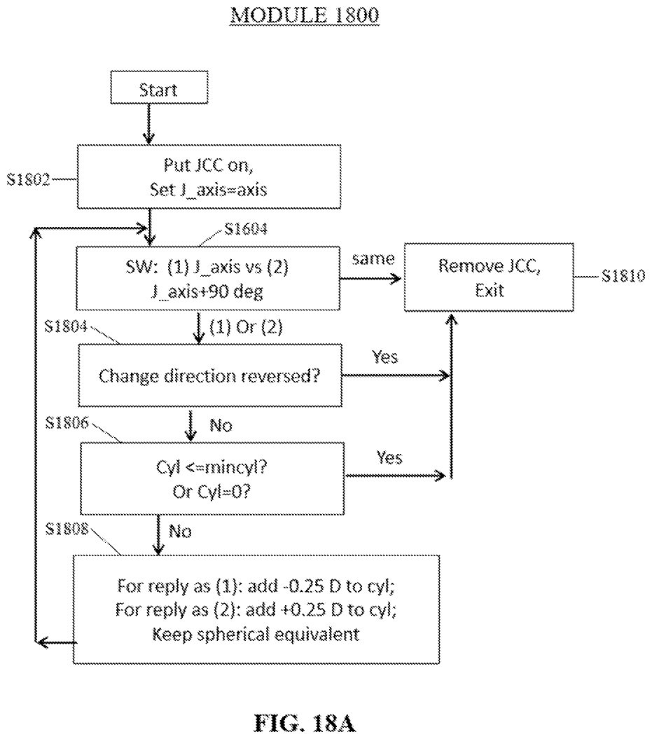

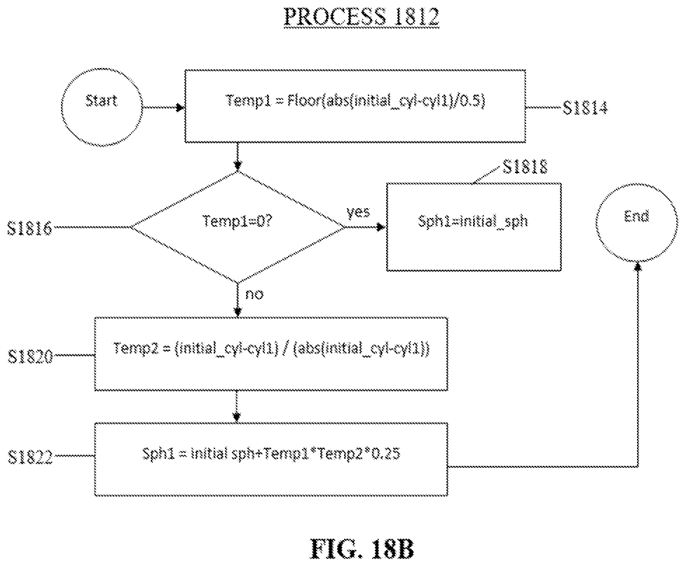

FIGS. 18A-18B illustrate flowcharts showing an exemplary cyl module, wherein FIG. 18A shows a cyl module and FIG. 18B shows a process for maintaining a spherical equivalent, in accordance with an embodiment of the present disclosure.

Cyl module 1800 may use the JCC to determine a cylinder value for the patient. In a step S1802, J_axis of the JCC is set to the current axis value of the cylinder lens. In a step S1804, two views are presented to the patient where (1) the JCC is set to J_axis and (2) the JCC is set to 90 degrees more than J_axis, and the patient is asked which view is clearer. The patient may be queried using first voice recognition module 1000 shown in FIG. 10 or second voice recognition module 1100 shown in FIG. 11. If the patient selects (1) or (2) and the change direction is reversed as determined in a step S1804, the JCC may be removed and cyl module 1800 exits in a step S1810. If the change direction is not reversed, in a step S1806, the cyl value is less than mincyl or the cyl value is equal to 0, cyl module 1800 may continue to S1810. If not, in a step S1808, the cyl may be adjusted by −0.25 D if the client indicated a preference for (1) or adjusted by +0.25 D if the client indicated a preference for (2). Further, the phoropter may adjust to maintain a spherical equivalence (i.e. balancing the change of the spherical lens and the cylinder lens). The process may then repeat from S1804. If the patient's first preference differs from the patient's second preference, the change direction may be determined to be reversed. For example, in the first round, the patient prefers view (1) and in the second round, the patient prefers view (2). Since the first preference is (1) and the 2nd preference is different from the initial preference, the change direction is reversed. In another example: in the first round, the patient prefers view (1). In the second round, the patient prefers view (1). In the 3rd round, the patient prefers view (2). Because the second preference is (1) and the third preference is different from the second preference, the change direction is reversed. The cylinder lens may be adjusted by removing the current cylinder lens inside the phoropter in front of the patient's eye and positioning another cylinder lens with corresponding to the adjusted cyl value. For example, the patient prefers view (1), it is determined that the change direction is not reversed, Cyl>mincyl and Cyl is not zero, the cyl value may be decreased by 0.25 D. If the current cylinder lens inside the phoropter has a power of −3.00 D, cyl module 1800 sends a command to the phoropter and the phoropter replaces the current cylinder lens with a cylinder lens which has power of −3.25 diopter. Cyl <=mincyl means that the software compares the current cylinder lens value to the mincyl value stored in a database for the current patient. For example, if the mincyl is equal to −4.00 D in the database and the current cylinder lens inside the phoropter is −3.00, the software determines that Cyl>mincyl.

Whenever the cylinder value is updated, the spherical value may need to be updated as well to maintain a spherical equivalent. Process 1812 may begin with a step S1814 wherein a first temporary variable (Temp1) may be used to store the result of Floor(abs(initial_cyl-cyl1)/0.5), wherein Floor(x) is the floor function (i.e. returns the greatest integer less than or equal to x), abs(x) is the absolute value function, initial_cyl is the initial cylinder value, and cyl1 is the updated cylinder value. If in a step S1816 it is determined that Temp1 is equal to 0, the updated sphere value (Sph1) remains the initial sphere value (initial_sph). If not, in a step 1820, a secondary temporary variable (Temp2) may be used to store the result of (initial_cyl-cyl1)/(abs(initial_cyl-cyl1)). In a step S1822, Sph1 may be set to initial_sph+Temp1*Temp2*0.25. Thus, Sph1 is updated according to the updated cylinder value, and the spherical equivalent is maintained.

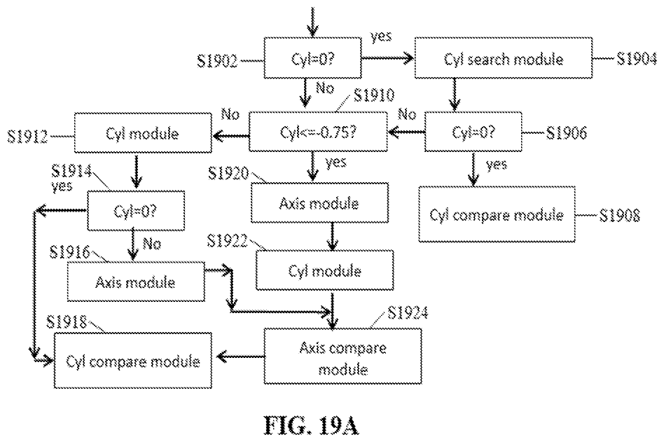

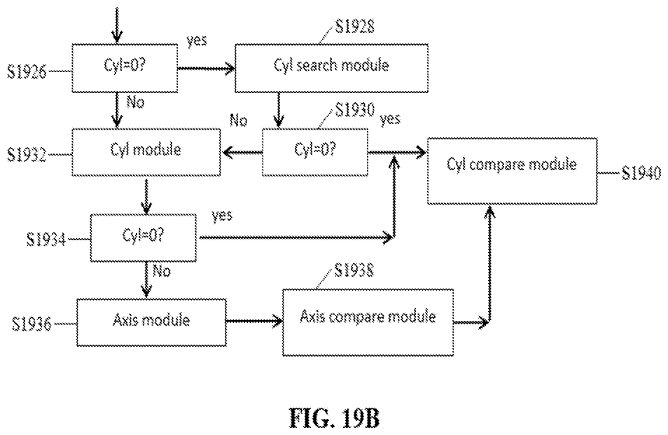

FIGS. 19A-19B illustrate flowcharts showing alternative cyl and axis paths, wherein FIG. 19A shows a first alternative path and FIG. 19B shows a second alternative path in accordance with an embodiment of the present disclosure. As shown, alternative method steps may be used in the automated refraction process shown in FIG. 5 above. Namely, steps S514-S526 between comparing the cyl value to 0 and the cyl compare module may be replaced with the steps shown in FIGS. 19A-19B.

With reference to FIG. 19A, In a step S1902, the current cyl value may be compared to 0. If the cyl value is equal to 0, the process may continue to a step S1904 wherein the cyl value may be adjusted according to the cyl search module. If the cyl is still 0, as determined in a step S1906, the process may continue to the cyl compare module in a step S1908. If not, in a step S1910, the cyl value may be compared to −0.75 D. If less than or equal to −0.75 D, the process may continue to the axis module in a step S1920, to the cyl module in a step S1922, to the axis compare module in a step S1924, and to the cyl compare module in a step S1918. If the cyl value is greater than −0.75 D, the process may continue to the cyl module in a step S1912. The cyl value may then be compared to 0 in a step S1914. If equal to 0, the process may proceed to the cyl compare module in S1918. If not equal to 0, the process may continue to the axis module in a step S1916, the axis compare module in S1924, and the cyl compare module in S1918.

With reference to FIG. 19B, if the cyl value is equal to 0 as determined in a step S1926, the process may continue to the cyl search module in a step S1928 and compared again to 0 in a step S1930. If in S1930 the cyl value is determined to be equal to 0, the process may continue to the cyl compare module in a step S1940. If not, the process may proceed to the cyl module in a step S1932. In a step S1934, the cyl value may be compared to 0. If the cyl value is 0, the process may continue to S1940. If not, the process may continue to the axis module in a step S1936, to the axis compare module in a step S1938, and to S1940.

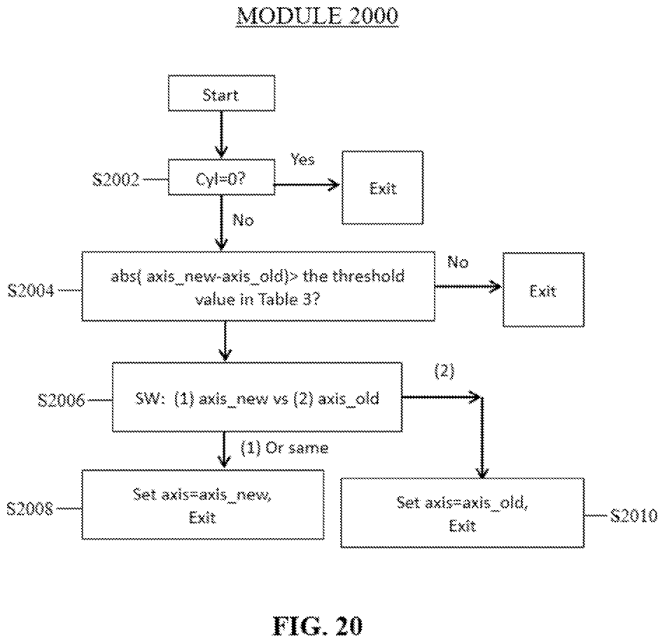

FIG. 20 illustrates a flowchart showing an exemplary axis compare module, in accordance with an embodiment of the present disclosure. Axis compare module 2000 may begin with a step S2002 wherein the cyl value may be compared to 0. If 0, the automated refraction process continues to the next module. If not, axis_old and axis_new are compared to the threshold value in Table 3 below according to abs(axis_new −axis_old) >=the threshold value in Table 3. The “axis_old” value is the axis angle of the most recent prescription on file for the patient or the measurement of the patient's current eyeglass prescription. If both are available, the most recent prescription may be used. The “axis_new” value is the axis value at the start of axis compare module 2000 (i.e. the axis value of the current cylinder lens inside the phoropter). If less than the threshold value, the automated refraction process may continue to the next module. If greater than or equal to the threshold value, in a step S2006, the patient may be presented two views using: (1) axis_new and (2) axis_old. When seeking responses from the patient, axis compare module 2000 may utilize first voice recognition module 1000, as described above with reference to FIG. 10. In the present embodiment, the expected responses are “(1)”, “same”, and “(2)”. If the patient says “(1)” or says “same”, in a step S2008, the axis is set to axis_new and axis compare module 2000 exits. If the patient says “(2)”, in a step S2010, the axis is set to axis_old and axis compare module 2000 exits. “abs” means the absolute value. When the axis is set to axis_new or axis_old, a command is sent to the phoropter and the phoropter sets the axis angle of the current cylinder lens inside the phoropter to axis_new or axis_old. The threshold value refers to the axis difference column in table 3.

TABLE 3

| Cyl Value | Axis Difference |
|---|---|
| −0.25 | 15 |
| −0.5 | 8 |
| −0.75 | 6 |
| −1, −1.25, −1.5 | 4 |
| <=−1.75 | 3 |

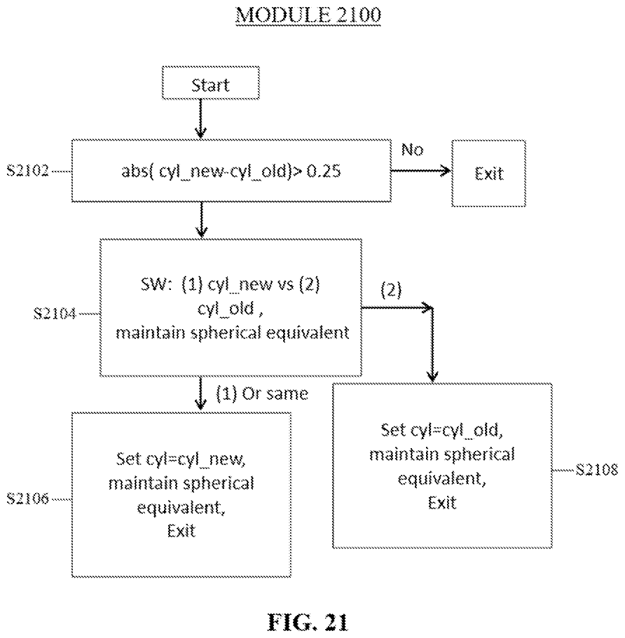

FIG. 21 illustrates a flowchart showing an exemplary cyl compare module, in accordance with an embodiment of the present disclosure. The “cyl_old” value is the cyl value of the patient's most recent prescription or the cyl value of the patient's current eyeglass prescription. If both are available, the most recent prescription may be chosen. The “cyl_new” value is the cyl value at the start of cyl compare module 2100. Cyl compare module 2100 uses the voice recognition process shown in FIG. 10. In a step S2102, cyl compare module 2100 may make a comparison using cyl_old and cyl_new according to: abs(cyl_new-cyl_old)>0.25. If less than or equal to 0.25 D, cyl compare module 2100 may exit. If greater than 0.25 D, the patient may be presented with two views using: (1) cyl_new and (2) cyl_old. After the adjustment of the cylinder lens power in the phoropter, the phoropter may adjust the value of the spherical lens in the phoropter based on a spherical equivalent principle. If the patient selects (2), in a step S2108, cyl may be set to cyl_old while maintaining spherical equivalency and cyl compare module 2100 may exit. If the patient selects (1) or indicates that the views are equally clear, in a step S2106, cyl may be set to cyl_new while maintaining spherical equivalency and cyl compare module 2100 may exit.

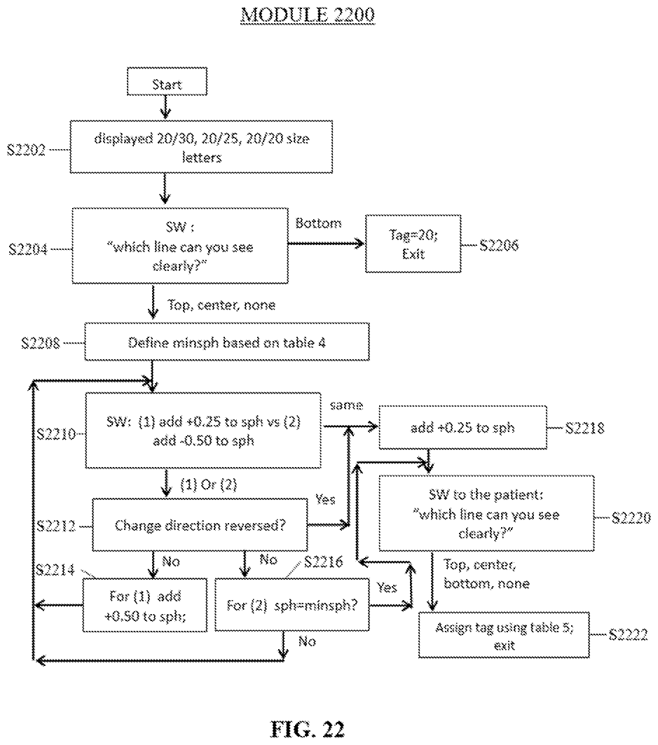

FIG. 22 illustrates a flowchart showing an exemplary optimization at 20/40 module, in accordance with an embodiment of the present disclosure. In optimization at 20/40 module 2200, in a step S2202, 3 lines of letters or words may be displayed to the patient: 20/30 size letters, 20/25 size letters, and 20/20 size letters. In the present embodiment, the display may display letters or words. The top line shows a few (5 or 6) letters or words with 20/30 size. The center line shows a few (5 or 6) letters or words with 20/25 size. The bottom line shows a few (5 or 6) letters or words with 20/20 size. There may be one (20/30 size) or more lines separation between each line. Optimization at 20/40 module 2200 may utilize second voice recognition module 1100 as shown in FIG. 11 in a step S2204, where the message: “Which line can you see clearly?” may be broadcast to the patient. Expected replies may include “top”, “bottom”, “center”, or “none”. If the patient responds “bottom”, in a step S2206 optimization at 20/40 module 2200 may exit. If the user responds “top”, “center”, or “none”, minsph may be defined based on table 4 in a step S2208, and in a step S2210 two views may be displayed to the patient: (1) a view with +0.25 D added to the sph value and (2) a view with −0.50 added to the sph value. Expected results may include “1”, “2”, or “same”. If the patient responds “same”, in a step S2218, 0.25 D may be added to the sph and the patient may again be asked “which line can you see clearly?” in a step S2220. In a step S2222, depending on the response from S2220, a tag may be assigned according to table 5 and optimization at 20/40 module 2200 may exit. If in S2210 the patient selects (1) or (2) and the change direction is reversed as determined in a step S2212, optimization at 20/40 module 2200 may continue to S2218. If not and (1) was selected, in a step S2214, 0.50 D may be added to the sph and optimization at 20/40 module 2200 may continue to S2210. If not and (2) was selected, in a step S2216, sph may be compared to minsph. If sph is equal to minsph, optimization at 20/40 module 2200 may continue to S2218. If sph is not equal to minsph, optimization at 20/40 module 2200 may continue to S2210. The change direction may be based on previous iterations of optimization at 20/40 module 2200. For example, in the first round, the patient prefers view (1). In the second round, the patient prefers view (2). Since the first preference is (1) and the second preference is different from the first preference, the change direction is considered reversed. In table 4, “sph” is the power of the spherical lens at the start of optimization at 20/40 module 2200. “Minsph” in table 4 may overwrite the “minsph” previously defined in the automated refraction process. Alternatively, the lesser value of minsph and Minsph may be chosen as the bottom threshold in optimization at 20/40 module 2200.

TABLE 4

| Patient Preference | Minsph |
|---|---|
| top | Minsph = sph − 0.75 |
| middle | Minsph = sph − 0.50 |
| bottom | Minsph = sph − 0.25 |
| none | Minsph = sph − 01.00 |

TABLE 5

| Patient preference | Tag |
|---|---|
| top | 30 |
| middle | 25 |
| bottom | 20 |
| none | 40 |

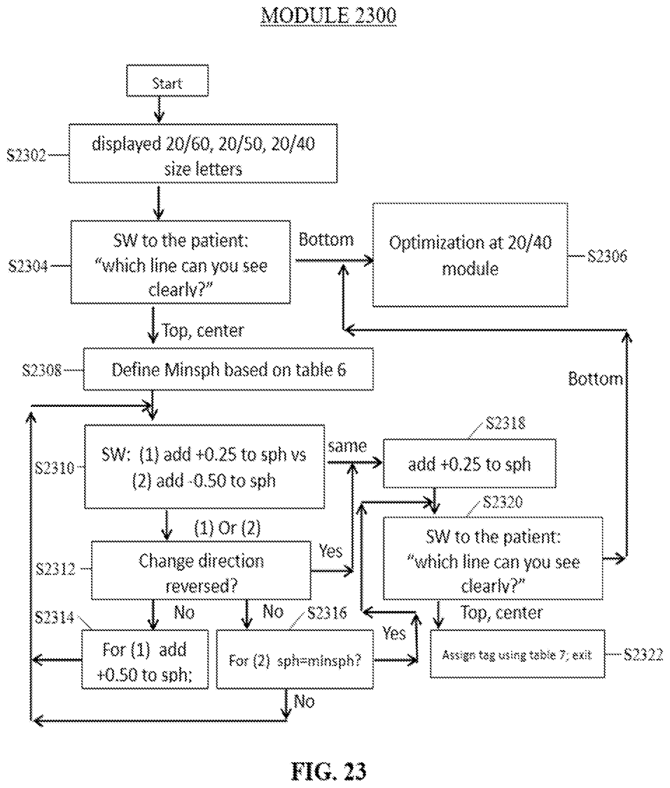

FIG. 23 illustrates a flowchart showing an exemplary optimization at 20/60 module, in accordance with an embodiment of the present disclosure. In optimization at 20/60 module 2300, in a step S2302, 3 lines of letters or words may be displayed to the patient: 20/30 size letters, 20/25 size letters, and 20/20 size letters. In the present embodiment, the display may display letters or words. The top line shows a few (5 or 6) letters or words with 20/30 size. The center line shows a few (5 or 6) letters or words with 20/25 size. The bottom line shows a few (5 or 6) letters or words with 20/20 size. There may be one (20/30 size) or more lines separation between each line. Optimization at 20/60 module 2300 may utilize second voice recognition module 1100 as shown in FIG. 11 in a step S2304, where the message: “Which line can you see clearly?” may be broadcast to the patient. Expected replies may include “top”, “bottom”, “center”, or “none”. If the patient responds “bottom”, in a step S2306 optimization at 20/60 module 2300 may proceed to optimization at 20/40 module 2100. If the user responds “top”, “center”, or “none”, minsph may be defined based on table 6 in a step S2308, and in a step S2310 two views may be displayed to the patient: (1) a view with +0.25 D added to the sph value and (2) a view with −0.50 added to the sph value. Expected results may include “1”, “2”, or “same”. If the patient responds “same”, in a step S2318, 0.25 D may be added to the sph and the patient may again be asked “which line can you see clearly?” in a step S2320. In a step S2322, depending on the response from S2320, a tag may be assigned according to table 7 and optimization at 20/60 module 2300 may exit. If in S2310 the patient selects (1) or (2) and the change direction is reversed as determined in a step S2312, the optimization at 20/60 module may continue to S2318. If not and (1) was selected, in a step S2314, 0.50 D may be added to the sph and optimization at 20/60 module 2300 may continue to S2310. If not and (2) was selected, in a step S2316, sph may be compared to minsph. If sph is equal to minsph, optimization at 20/60 module 2300 may continue to S2318. If sph is not equal to minsph, optimization at 20/60 module 2300 may continue to S2310. The change direction may be based on previous iterations of optimization at 20/60 module 2300. For example, in the first round, the patient prefers view (1). In the second round, the patient prefers view (2). Since the first preference is (1) and the second preference is different from the first preference, the change direction is considered reversed. In table 6, “sph” is the power of the spherical lens at the start of optimization at 20/60 module 2300. “Minsph” in table 6 may overwrite the “minsph” previously defined. Alternatively, the lesser value of minsph and Minsph may be chosen as the bottom threshold in optimization at 20/60 module 2300.

TABLE 6

| Patient Preference | Minsph |
|---|---|
| top | Minsph = sph − 1.25 |
| middle | Minsph = sph − 1.00 |
| bottom | Minsph = sph − 0.75 |

TABLE 7

| Patient preference | Tag |
|---|---|
| top | 60 |
| middle | 50 |
| bottom | 40 |

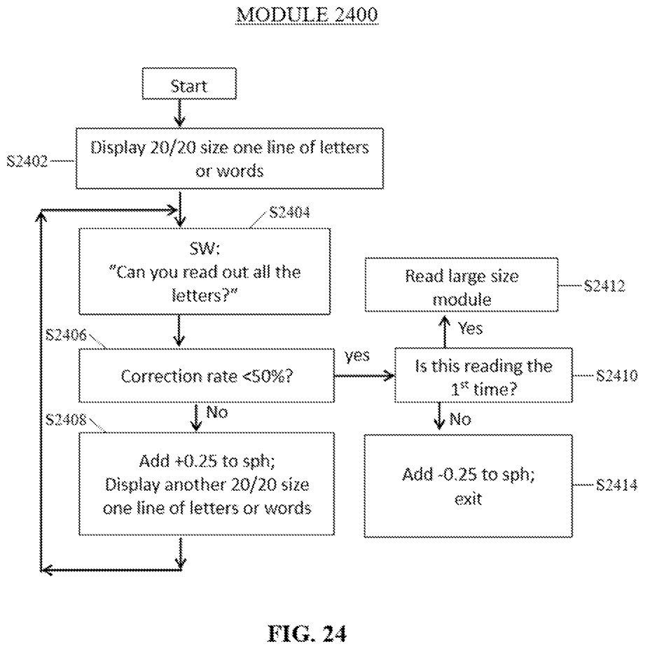

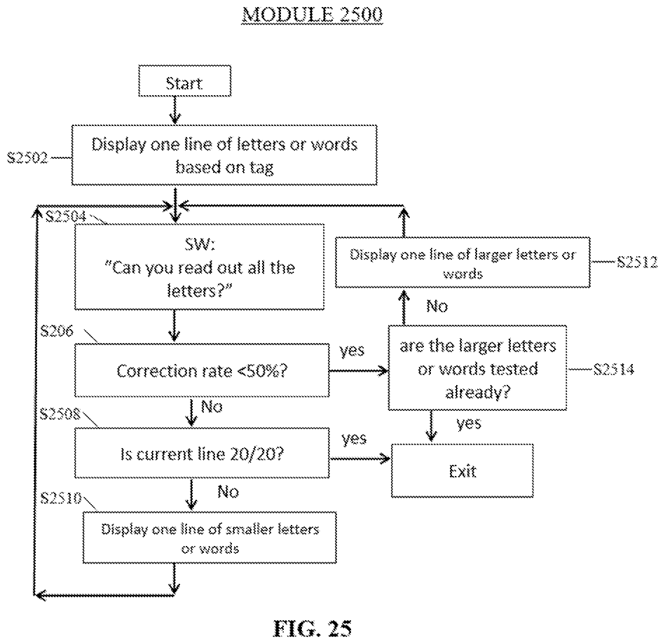

FIG. 24 illustrates a flowchart showing an exemplary read 20/20 size module, in accordance with an embodiment of the present disclosure. If, in optimization at 20/40 module 2200, the tag value is 20, automated refraction process 700 may proceed to the read 20/20 size module. If not, automated refraction process 70 may continue to read large size module 2500 of FIG. 25.

Read 20/20 size module 2400 utilizes second voice recognition module 1100 as shown in FIG. 11. In a step S2402, a line of letters or words of 20/20 size may be displayed to the patient. In a step S2404, the message “Can you read out all the letters?” is broadcasted to the patient. Once the patient reads out the letters or words shown on the display, the software compares the return from second voice recognition module 1100 with the letters or words shown on the display and calculates the correction rate. Read 20/20 size module 2400 controls what kinds of letters or words shown on the display. If the correction rate is greater than 50%, in a step S2408, 0.25 is added to the sph, a different line of 20/20 sized letters or words are shown to the patient, and read 20/20 size module 2400 continues to S2402. S2408 may effectively remove an accommodation effect which is more pronounced in younger individuals. If the correction rate is less than 50% and if it is the first iteration of reading the letters or words as determined in a step S2410, read 20/20 size module 2400 may exit and continue to read large size module 2500. If it is not the first iteration of reading the 20/20 size letters or words, 0.25 D may be detracted from the sph and read 20/20 size module 2400 may exit. At the end of read 20/20 size module 2400, there may be a number of different outcomes: exiting to read large size module 2500, the patient's vision in the current eye is determined to be 20/20− when the correction rate is higher or equal to 50% but less than 100%, or the patient's vision is determined to be 20/20 when the correction rate is 100%.

FIG. 25 illustrates a flowchart showing an exemplary large size module, in accordance with an embodiment of the present disclosure. Read large size module 2500 may use a tag value to determine the size of letters or words shown on the display. For example, if the tag value is 30, the software shows one line of 20/30 size letters or words on the display. In a step S2502, a line of letters or words is displayed to the patient, where the size of the letters or words depends on the tag value. For example, the patient is displayed 20/30 size letters on the display if the patient's tag value is 30. In a step S2504, the message “Can you read out all the letters?” may be broadcasted to the patient. In a step S2506, the correction rate of the patient's response is calculated. If the software determines that the correction rate is higher than 50%, and the current line does not use 20/20 size letters, as determined in a step S2508, a line of letters or words with smaller sizing may be displayed to the user and read large size module 2500 may continue to S2504. For example, a line of 20/25 size letters or words may be displayed if the previous line used 20/30 size letters or words. If in S2508 the current line is 20/20, read large size module 2500 may exit. If in S2506 the correction rate is less than 50%, read large size module 2500 may continue to a step S2514 to determine if larger letters or words have already been tested. If so, read large size module 2500 may exit. If not, in a step S2512, a line with larger letters or words may be displayed to the patient and read large size module 2500 may continue to S2504.

In another example, initially the patient may be presented 20/30 size letters and asked to read the line of letters. The patient may have a difficult time in reading the letters. The software then determines that the correction rate is less than 50%. In this example, larger letters or words (for example 20/40 size letters or words) were not previously shown to the patient. Hence the software shows one line of 20/40 size letters or words on the screen and asks the patient to read the 20/40 size letters or words.

In yet another example, initially the patient may be presented a line of 20/30 size letters on the display and asked to read the letters. The software may then determine that the correction rate is higher than 50%. A line of 20/25 size letters or words may be displayed and the patient may be asked to read the new line of letters. The software then determines that the correction rate is lower than 50% for the 20/25 size letters or words. Here the larger letters or words (namely 20/30 size letters and words) have already been tested. The software then exits the module. Assuming the correction rate for the 20/30 size letters or words is 75%, the software determines that the DVA cc (distant visual acuity with correction) of this eye is 20/30-.

The following is the sequence of letter sizes from small to large: 20/10, 20/15, 20/20, 20/25, 20/30, 20/40, 20/50, 20/60, 20/70, 20/80, 20/100, 20/125, 20/150, 20/200, 20/400, 20/800, 20/1000. For example: the display current displays letter size of 20/40. For example, in S2510, the current letters of 20/40 size may be replaced with a new line of letters of 20/30 size. In S2512, for example, the display current displays letter size of 20/50. The current letters of 20/50 size may be replaced with a new line of letters of 20/60 size. By combining the correction rate with a corresponding letter size on the display, DVA cc may be determined. For example, the correction rate is 60% when the patient is reading the 20/40 size words on the display and the correction rate is 40% when the patient is reading the 20/30 size words on the display. The DVAcc in this scenario is 20/40-. In another example, the correction rate is 100% when the patient is reading the 20/25 size words on the display and the correction rate is 30% when the patient is reading the 20/20 size words on the display. The DVAcc is determined to be 20/25.

If the DVAcc for any eye is worse than 20/20-, the automated refraction process may continue to near vision test module 2700. Otherwise, the automated refraction process may continue to binocular balance module 2600.

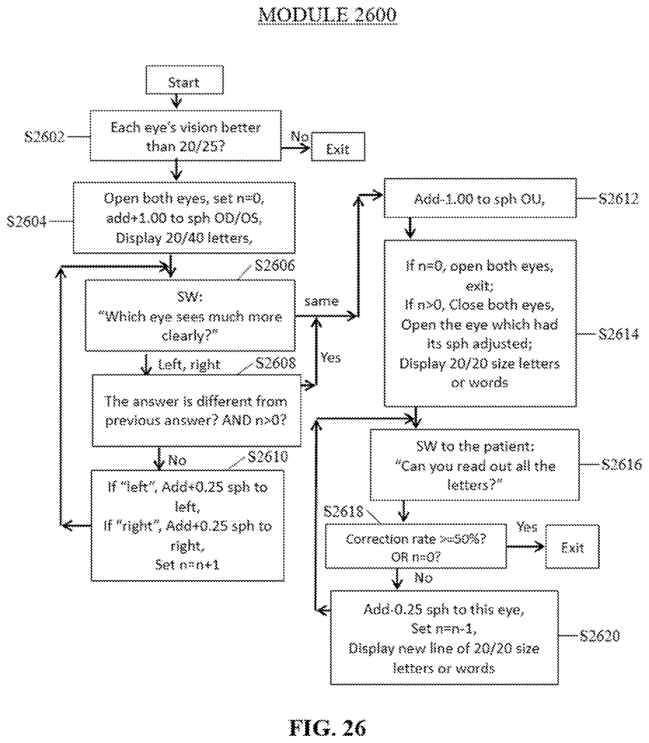

FIG. 26 illustrates a flowchart showing an exemplary binocular balance module, in accordance with an embodiment of the present disclosure. “OD” may refer to the right eye. “OS” may refer to the left eye. Binocular balance module 2600 uses first voice recognition module 1000 as shown in FIG. 10. In the present embodiment, n may be a variable used to track a number of iterations of adjusting sph values in binocular balance module 2600. In a step S2602, if the prescription for each eye is not better than 20/25, binocular balance module 2600 may exit. If better than 20/25, in a step S2604, both eyes may be open, n is set to 0, 1.00D is added to the sph of each eye, and 20/40 size letters may be displayed. If the patient determines that the left eye is clearer than the right eye or that the right eye is clearer than the left eye as determined in S2606, binocular balance module 2600 may continue to a step S2608 wherein it may be determined whether or not to increase the sph value for one of the eyes. If, in a previous iteration, the patient answered that one eye (e.g. the right eye) is clearer and in the current iteration the patient answered that the other eye (e.g. the left eye) is clearer, binocular balance module 2600 may continue to a step S2612. If not (i.e. the patient's answer in the previous iteration was the same as the current iteration), in a step S2610, 0.25 may be added to the sph value of the clearer eye, the n value may be increased by 1, and the process may loop back to S2606. If the patient determines that the left and right eyes are equally clear, binocular balance module 2600 may continue to S2612, wherein −1.00 D may be added to the sph for both eyes. In a step S2614, if n is 0, automated refraction process 700 may exit binocular balance module 2600 and continue to the next module. If n is greater than 0, the patient may be instructed to open the eye with an adjusted sph value and 20.20 size letters or words may be displayed. In a step S2616, the message “Can you read out all the letters?” may be broadcasted to the patient, and the correction rate of the patient's response may be recorded. In a step S2618, if the correction rate is greater or equal to 50% or the n value is equal to 0, binocular balance module 2600 may exit. If not, −0.25 D may be added to the sph value of the current eye, n may be decreased by 1, and a new line of 20/20 size letters or words may be displayed to the user before looping back to S2616. At the end of binocular balance module 2600, the software combines the sph, cyl, axis setting in the phoropter with the correction rate to generate a report of patient's distance visual prescription and distance visual acuity.

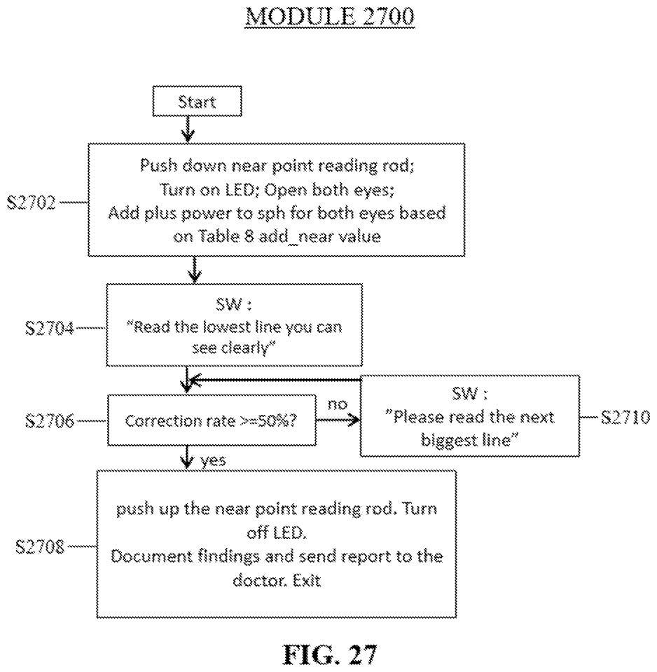

FIG. 27 illustrates a flowchart showing an exemplary near vision test module, in accordance with an embodiment of the present disclosure. In a step S2702 of near vision test module 2700, a near point reading rod may be initialized, an LED may be used to illuminate a reading card of the near point reading rod, and the sph value for each eye may be adjusted according to the add_near value (i.e. an updated add value) in Table 8. As shown in table 8, the add_near value may be determined based on the patient's age, and different values and age ranges may be used to determine the patient's updated add value. Table 8 may also be stored as a reference chart stored on a database and accessible by near vision test module 2700. An automated or manual near point reading rod may be used for near vision test module 2700. When using the automated near point reading rod, the control unit may determine that the near point reading rod is to be placed into the active position and sends a signal to actuate a motor connected to the near point reading rod such that the reading card of the near point reading rod is placed in front of the patient. Alternatively, the near point reading rod may be manually initialized, wherein the near point reading rod may be placed into the active position by, for example, without limitation, an assistant, a technician, a doctor, an optometrist, the patient, etc. Near vision test module 2700 may be used for patients with presbyopia, or the loss of near focusing ability that occurs with age. For example, without limitation, assume that the spherical lenses in the left eye and the right eye are initially set to −2.00 D and −3.00 D, respectively, and the patient is 55 years old. From table 8, the add_near for the current scenario is 2.25. Therefore, 2.25 is added to the spherical lens values of the left and right eyes, resulting in spherical lens values of +0.25 D and −0.75 D. The phoropter may then be adjusted to replace the current spherical lens for the left eye with a new spherical lens of power+0.25 D and the current spherical lens for the right eye with a new spherical lens of power −0.75 D. Near vision test module 2700 uses the voice recognition process shown in FIG. 11. In a step S2704, the message “Read the lowest line you can see clearly” may be broadcasted to the patient, and the patient's response may be recorded. Multiple lines of letters, words, shapes, etc. may be shown the patient using the reading card of the near point reading rod. Each of the lines may be different such that the response from the patient may be matched with a particular line and a correction rate may be determined based on the patient's response. If the correction rate is greater than or equal to 50% as determined in a step S2706, the message “Please read the next bigger line” may be broadcasted to the patient and the patient's response may be recorded. If in S2706 the correction rate is less than 50%, the reading rod may be stowed away, the LED may be turned off, and results may be recorded on the database in a step S2708. For example if (1) the patient was reading the 20/20 line on the near point reading card and (2) the correction rate is 100%, the patient's near vision with correction for both eyes (NVA cc OU) is recorded as 20/20. If in S2706 the correction rate is greater than or equal to 50%, in a step S2710, the message “Please read the next biggest line” may be broadcasted to the patient and near vision test module 2700 may loop back to S2706. The near point reading card may be made of paper and may have several lines of letters or words. Each line has a different size. Alternatively, the near point reading card may be an electronic display which is controlled by the control unit and may communicate with near vision test module 2700. Near vision test module 2700 may send commands to the near point reading card to change the letters or words displayed on it and change the size of the letters and words. It should be appreciated that table 8 shows one means for determining the add_near value, and other means for determining the add_near value may also be used.

TABLE 8

| Age | add_near |
|---|---|
| 40 < age <= 45 | 1.25 |
| 45 < age <= 50 | 1.5 |
| 50 < age <= 52 | 1.75 |
| 52 < age <= 55 | 2.0 |
| 55 < age <= 57 | 2.25 |
| age >= 57 | 2.50 |

If it is determined that the patient has diplopia, a prism test module may be used to determine the patient's prism value. In the other embodiment, the software may notify a doctor so that the doctor can perform further vision testing. The prism test module may include the following steps:

Step 1: The patient may be instructed to open both eyes, and a prism value may be added to the existing distance refraction results. A prism power and the base axis angle may be determined by Sheard's criterion. The prism power and the base axis angle may also be determined by Percival's criterion. The prism power and the base axis angle may also be extracted from an existing file in the EHR. The prism power and the base axis angle may also be extracted from the patient's most recent eyeglass prescription.

Step 2: If the client has far diplopia, the letters or words on the display at a far distance may be used as the visual target for the patient. If the client has near diplopia, the near point reading card may be used as the target for the patient. If the patient has diplopia at both near and far ranges, the near point reading card may be used as the target for the patient. When the near point reading card is in use, the near point reading card may be pushed down and The LED may be turned on to illuminate the near point reading card.

Step 3: The display may show targets using letters, with the smallest letters corresponding to the patient's best corrected vision. For example: 4 lines of letters may be shown with sizes of 20/40, 20/30, 20/25, and 20/20 where the patient has a best corrected vision of 20/20.

Step 4: The near point reading card may show targets using letters, with the smallest letters corresponding to the patient's best corrected vision. For example: 4 lines of letters may be shown with sizes of 20/40, 20/30, 20/25, 20/20 where the patient has a best corrected vision of 20/20. The near point reading card may be made of paper; the near point reading card may also be an electronic display.

Step 5: The message “Can you read the letters clearly and comfortably?” may be broadcasted to the patient via a speaker.

If the reply is “yes”, the prism test module may document the prism values and the base axis angle of the prism value, sends the result to the doctor, and exit.

If the reply is “no”, the prism test module may add 0.5 prism diopter to each eye and loop back to step 5. Then if the reply is “yes”, go to step 6. If the reply is “no” again, the software may subtract 0.5 prism diopter from each eye and loop back to step 5. If the reply is “yes”, the prism test module may continue to step 6. The prism test module may continue looping back to step 5 until either the reply is “yes” or the prism power value is zero. When the prism power value is zero, the software may send a report to the doctor and remind the doctor that they need to perform the prism testing.

Step 6: The prism test module may document the settings of the prism and the corresponding base axis angle, send the report to the doctor, and exit.

The phoropter may be configured to use the following modes: (a) Mode A: the phoropter is fully controlled by the optometrist or medical staff and the optometrist adjusts the components in the phoropter while having professional conversation with the patient. In this mode, the optometrist is working in a traditional professional role to measure the refractive errors of the patient's eyes. (b) Mode B: The phoropter and the control unit may communicate with the patient and make decisions based on the patient's response. The optometrist is not in the same room with the patient. The optometrist may monitor the progress of the refraction via video through a wireless or wired connection. The optometrist may at any time may interrupt and stop the refraction by pressing a “stop” button using, for example, a computer, tablet, cell phone etc.; the phoropter may stop and send a progress report to the optometrist. The optometrist may take control of the phoropter and continue the refraction. (c) Mode C: The phoropter and the control unit may communicate with the patient and make decisions based on the patient's response. The optometrist is in the same room as the patient. The optometrist initially does not participate the refraction process. At any point, the optometrist may interrupt and stop the refraction process by pressing the “stop” button on a computer, tablet, cell phone etc. and the phoropter may stop and send a progress report to the optometrist. The optometrist may take control of the phoropter and continue the refraction examination. The optometrist may also use hand gestures or simply say “stop” to stop the phoropter from doing the refraction.

The saved audio files may be examined and the interpretation of the audio files may be compared with the output from the voice recognition module. The comparison results may be input into the voice recognition module, which may boost the recognition rate and reduce the error rate of recognizing the patient's speech.

The software may deliver the refraction results to the doctor. Once the refraction is finished, the software may send a message to the doctor. The doctor may read the message using a computer, cell phone, tablet, etc.

In case the software cannot communicate with the patient or the patient cannot continue the refraction, the software may notify the assistant and/or the doctor to intervene.

The software may notify the assistant and/or the doctor that the refraction is finished or stopped via a light. Once the refraction is finished or stopped, the software may send a signal to a light bulb hanging on the door of the examination room. The light bulb may flash or turn on. When the assistant and/or the doctor sees the light bulb flashing, they know that the refraction is finished or stopped.

The software may notify the assistant and/or the doctor that the refraction is finished or stopped via sound. Once the refraction is finished, the software may send a signal to a speaker in the medical office. The speaker may broadcast a message and tell the assistant and/or the doctor that the refraction is done or stopped.

An assistant may be in the general proximity of the phoropter. Once the refraction process is in the “Near vision test module”, the software may broadcast a message via a speaker, telling the assistant to show the patient the letters or the words on the near point reading card by pushing down a reading rod or simply holding the near point reading card in front of the phoropter. Once the software exits the “Near vision test module”, the software may broadcast a message via speaker telling the assistant to remove the near point reading card.

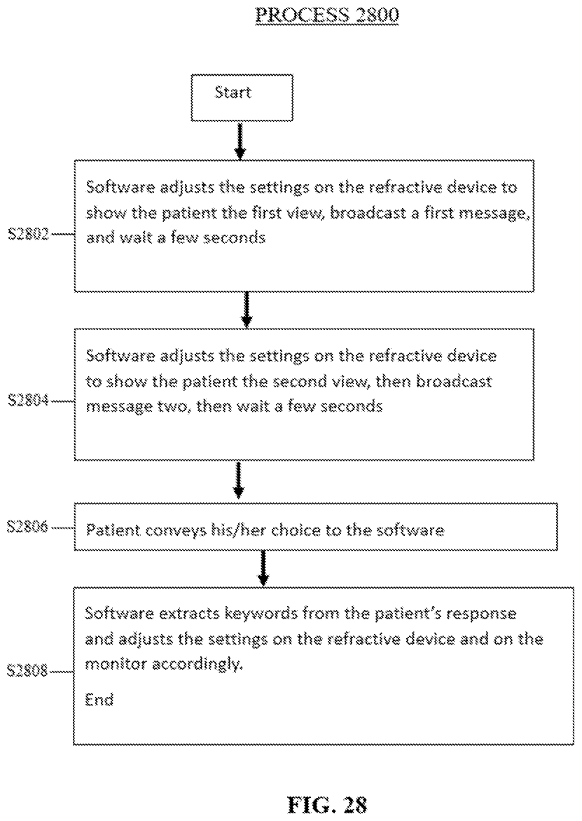

In autonomous refraction, the optometrist does not need to conduct the entirety of the refraction process. FIG. 28 illustrates a flowchart showing an exemplary automated refraction process, in accordance with an embodiment of the present disclosure. In the present embodiment, a speaker is used to broadcast the messages to the patient to tell the patient to compare the two different views and orally respond. Messages broadcasted by the speaker can also be displayed on a monitor or through virtual reality. The patient can then indicate a response. The patient can also use hand motions to respond to the system. A camera may be used to capture images of the patient's hand motions and software may use image analysis to analyze the images. The output of the image analysis may be used to make necessary adjustments to the mechanical and/or optical components of the refracting device and the displayed content on the monitor.

It should be noted that the patient can make verbal responses to the automated refraction process. The patient can also use an electrical device to indicate a choice. For example, without limitation, the patient can press “1” on a keyboard to indicate a preference for a first view. The patient can also use a joystick to indicate a choice. For example, without limitation, the patient can move the joystick to the left to indicate a preference for a first view. The electrical device may communicate with the software.

In a step S2802 of automated refraction process 2800, software may be used to adjust settings on a refractive device to show a patient a first view, broadcast a first message, and allow the patient to observe the first view. In a step S2804, the software may adjust the settings of the refractive device to show the patient a second view and wait for a response from the patient. In a step S2806, the patient may convey a choice between the first view and the second view to the software. In a step S2808, the software may extract keywords from the patient's response and adjust the refractive device accordingly. The first message may be used to inform the patient that the patient is being shown the first view, and the patient will need to compare the first view to a second view. For example, the first message may be “We will ask you to compare two views. Tell us which view is clearer. This is view 1”. The second message may be used to inform the patient that the patient is being shown the second view, and the patient will need to compare the second view to the first view. For example, message 2 may be “This is view 2. Please tell us which view is clearer. You can begin now. Or you can say repeat, we will repeat a few times for you”. If the patient says “repeat”, the aforementioned inquiry may be repeated. If the software cannot understand the patient's reply or the patient's reply is irrelevant to the inquiry, the inquiry will be repeated several times. The software may also use different terminology when referring to the first view and the second view. For example, without limitation, “image 3” or “image 4” may be used to refer to the first view and the second view, respectively.

The software may make decisions based on responses from the patient. A voice recognition module is deployed to translate an audio file from the patient to a text file. The voice recognition module may be incorporated within the software. The voice recognition module may alternatively be integrated in the DPSU and communicates with the software. Keywords may be “image 1”, “image 2”, “view 1”, “view 2”, “blurry”, “repeat” etc. The software may match responses from the patient with the keywords. The keywords may be “image” followed by a number, “view” followed by a number, etc.

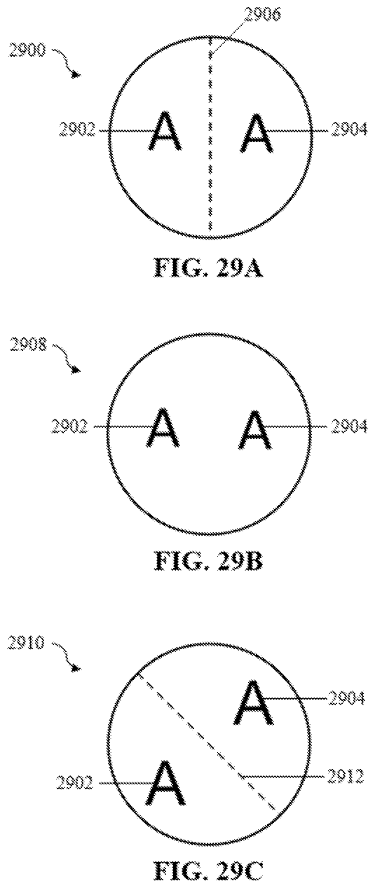

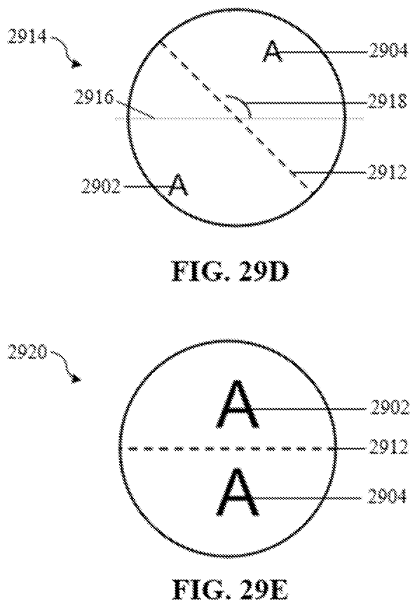

FIGS. 29A-29E illustrate exemplary views to be shown to a patient, wherein FIG. 29A shows a first view, FIG. 29B shows a second view, FIG. 29C shows a third view, FIG. 29D shows a fourth view, and FIG. 29E shows a fifth view, in accordance with an embodiment of the present disclosure Two views may be displayed to the patient at separate times, i.e. a timely sequence. The multiple (e.g. two) views may also be displayed to the patient in different locations than previous views. For example, without limitation, the refracting device may display a visual target (such as a letter, a pattern, words etc.) to different parts of the visual field of the patient, as shown in FIG. 29A. When the patient looks through the refracting device, the patient may perceive two images, while one image is displayed on the monitor (i.e. the two A's of FIG. 29A). The refracting device, i.e. phoropter, splits the light emitted from the monitor into two beams and takes each beam through different paths (each path with a different sets of optical components). Alternatively, the light beams may travel down a same path with different orientations, and two separate views may be visible to the patient at the same time. In another embodiment, a line (or dashed line) in the center of the patient's visual field may be used to remind the patient that there are two views present, as shown in FIG. 29A. FIG. 29E illustrates two views shown to the patient where the first view is at a top portion and a second view is at a bottom portion. FIG. 29B illustrates two views shown to the patient where a first view is at a left portion and a second view is at a right portion. Additionally, the two views can be oriented at any direction. FIG. 29C shows a first view at an upper right portion and a second view at a lower left portion. FIG. 29D shows a dividing line angle. The dividing line angle ranges from zero degree to 179 degree. Note that the horizontal line in the FIG. 29D may not be in the patient's field of view and is simply used to show the dividing line angle.

If there is no line separating the two views, the refracting device may rely on a specific angle setting or space position information to provide relative positions information of the two views shown to the patient. For example, the refracting device can send the following information to the software: “view 1 left; view 2 right”. When the software receives the reply from the patient and the reply is “left”, the software can determine that the patient prefers view 1.

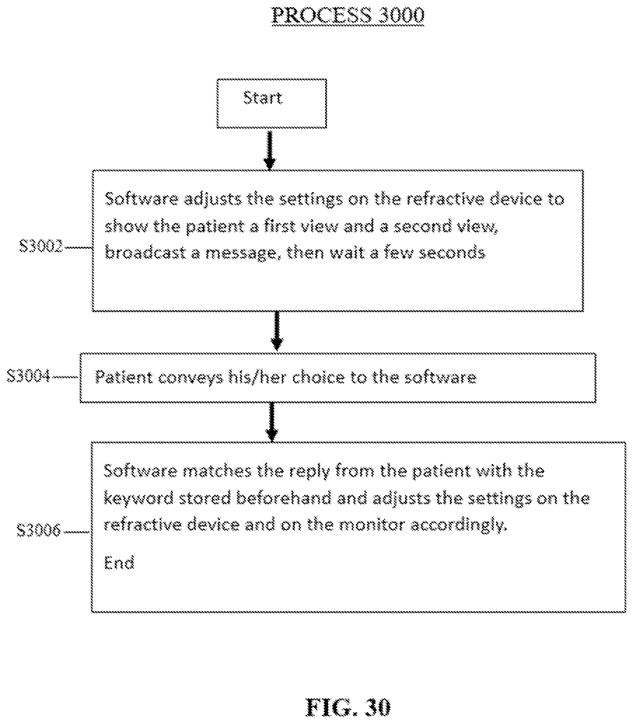

FIG. 30 illustrates a flowchart showing presentation of different views to a patient, in accordance with an embodiment of the present disclosure. As shown, the patients may be presented with different views, such as the views shown in FIGS. 4A-4C. In a step S3002 of process 3000, the software may adjust the settings of the refractive device to show the patient a first view and a second view, broadcast a message, and give the patient time to examine the different views. In a step S3004, the patient may convey a choice to the software. In a step S3006, the software may extract keywords from the patient's reply and adjust the settings of the refractive device and monitor accordingly.

The refracting device can control how the first view and the second view are displayed to the patient. As such, the refracting device can position the two views at different locations in the patient's field of view. Additionally, the refracting device may adjust the dividing line angle. In an alternative embodiment, the monitor or the DPSU may control how the views are displayed to the patient. In one embodiment, the refracting device controls the dividing line angle and sends dividing line angle information to the DPSU. The software utilizes the dividing line angle information as an input and to find corresponding key words pre-loaded into the software. Table 9 lists the key words corresponding to the dividing line angle. If the patient says “repeat”, the previous query will be repeated. If the software cannot understand the patient's reply or the patient's reply is irrelevant to the inquiry, the inquiry will be repeated several times. A voice recognition module may translate the audio file into text. The software then matches the text file with the key words and to determine the patient's preferred view. After the preferred view is determined, The software may adjust the settings of the monitor and the refracting device.

TABLE 9

| Dividing line angle | Key words |
|---|---|
| 0 to 10 deg | Top, bottom |
| 11 to 80 deg | Top left, bottom right, top, |
| | bottom, left, right |
| 81 to 100 deg | Left, right |
| 101 to 169 deg | Top right, bottom left, top, |
| | bottom, left, right |
| 170 to 179 deg | Top, bottom |

For example, the patient may be presented with the message: “We will ask you to compare two views. Both views are shown to you at the same time. Tell us which view is clearer. You can say: top view, bottom view, or repeat”.

The two views may be separated and have the same symbol. In an alternative embodiment, two views may be presented to the patient at difference locations where each view has unique symbols. The symbols may include, but are not limited to letters, images, drawings, sentences, words, pictures, etc.

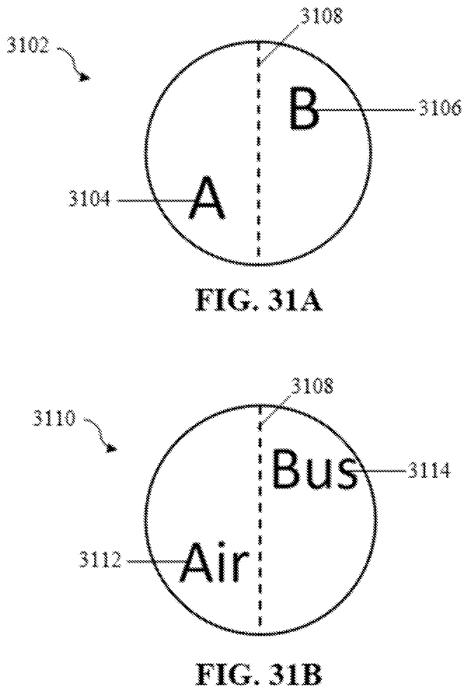

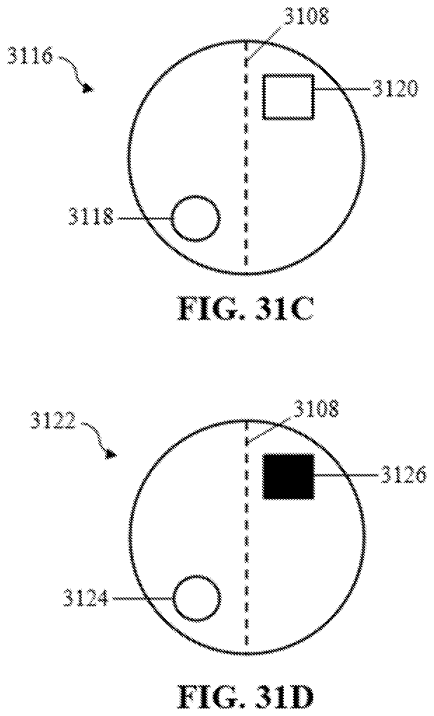

FIGS. 31A-31D illustrate exemplary patterns to be shown to a patient, wherein FIG. 31A shows a first pattern, FIG. 31B shows a second pattern, FIG. 31C shows a third pattern, and FIG. 31D shows a fourth pattern, in accordance with an embodiment of the present disclosure. Different types of views are shown. In one embodiment, different views with different colors may be shown to the patient. As such, the software expects a patient to indicate a preference by describing the characteristics of the view the patient prefers, such as, but not limited to, color, shape, word, letter etc. For example, the message may be: “We will ask you to compare two views. Both views are shown to you at the same time. Tell us which view is clearer. You can say: A, B, or repeat”. In another example, the message may be: “We will ask you to compare two views. Both views are shown to you at the same time. Tell us which view is clearer. You can say: Air, Bus, or repeat”. In another example, the message may be: “We will ask you to compare two views. Both views are shown to you at the same time. Tell us which view is clearer. You can say: square, circle, or repeat”. In another example, the message may be: “We will ask you to compare two views. Both views are shown to you at the same time. Tell us which view is clearer. You can say: black, white, or repeat”. The key words are stored in the software and may vary depending on the type of view shown. For example, the keywords may be (A, B, repeat), (square, circle, repeat), (Air, Bus, repeat), (black, white, repeat). The software then compares the patient's reply to the key words and determines which view (e.g. view 1 or view 2) that the patient prefers.

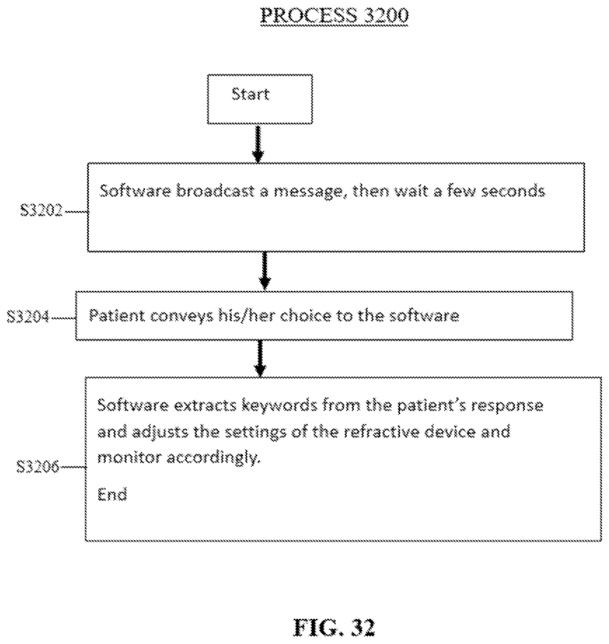

FIG. 32 illustrates a flowchart showing a second voice recognition process, in accordance with an embodiment of the present disclosure. In a step S3202 of process 3200, the software may broadcast a message and the client may be given time to observe the views shown. In a step S3204, the patient may convey a choice to the software. In a step S3206, The software may extract keywords from the patient's response and adjust the settings of the refractive device and monitor accordingly. The message may be used to inform the patient that the patient will need to report if they can see the views clearly. For example, the message may be: “Tell us if you can see these letters clearly. You can say: yes, or no”. The expected keywords may vary. For example: (yes, no).

The message may also be used to inform the patient that patient will need to report the exact letters or words or pictures he/she sees. For example, the monitor shows letters “a k d h m”; the message may be “Can you read out the letters you see?” The expected keywords are thus (a k d h m). The software then compares the reply from the patient to the keywords and proceeds to the next round of inquiry.

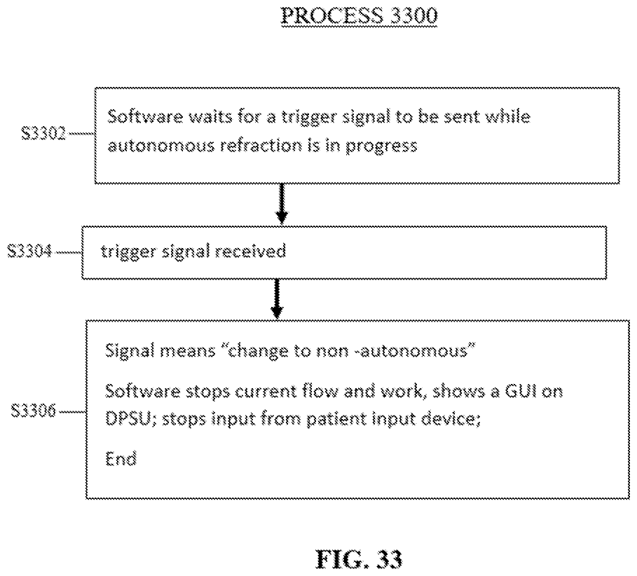

FIG. 33 illustrates a flowchart showing a transition from autonomous refraction examination to non-autonomous refraction examination, in accordance with an embodiment of the present disclosure. In a step S3302 of process 3300, the software may wait for a trigger signal to be sent while autonomous refraction is in progress. In a step S3304, the trigger signal may be received. The trigger signal may be an electrical signal generated when the optometrist or staff presses a button. Alternatively, the trigger signal may be generated by a voice command from the staff or optometrist. When the trigger signal is detected, in a step S3306, the software stops the current workflow and shows a GUI (graphic user interface) on the DPSU (e.g. the screen of a computer where the computer is used as the DPSU). The GUI includes buttons and/or icons and the buttons and/or icons may be used to control of the monitor, refracting device, and speaker. The patient input device may also be configured to stop functioning after the trigger signal is received.

In the present disclosure, a display may be used to show the patient letters and or words or videos or images. The software controls the display via a wired or wireless connection. The display may be, for example, without limitation, a television, a monitor, a projector, etc. The software may send a command to the control unit to display letters, words, etc. as needed. As the voice recognition modules may have a higher accuracy rate when processing audio files for a patient reading out words compared to the audio files with the patient reading out letters, words may be displayed to the patient to increase the accuracy of the voice recognition modules. Alternatively, images (e.g. shapes) may be shown to the patient instead of letters or words.

In another embodiment, a camera may be positioned in front of the patient. The distance between the camera and the patient may be set depending on various criteria (e.g. at 1 meter). As such, input may still be recorded from patients with speaking disabilities or patients who do not wish to speak. Patients can elect to use hand gestures to indicate a response to a message broadcasted through the speaker. For example, the patient can show one finger to indicate that the patient prefers image one. The camera then records the hand gesture and sends the images/videos to the control unit. The camera is connected to the control unit via a wired or wireless connection. The software performs image analysis, interpreting the patient's reply and performing hardware adjustments accordingly.

For patients with hearing disabilities or deficiencies, the display may be used to show a question. A line may be used to separate the target (i.e. letters, words, images, etc.) and the broadcasted question. In one embodiment, the question is displayed on the bottom half of the display. The letters/words serving as the visual target are displayed at the top half of the display. The patient may also be given a longer period for response to the question displayed.

FIG. 32 illustrates a flowchart showing an exemplary automated refraction process, in accordance with an embodiment of the present disclosure. In a step S3202, patient information may be obtained from the patient and initial eyeglass prescription values may be chosen from the patient information. The patient information may include, for example, without limitation, a current eyeglass prescription, a last eyeglass prescription on file, auto refractor data, prism data (if applicable), etc. In one embodiment, the initial eyeglass prescription values may be propagated from the current eyeglass prescription. In another embodiment, the initial eyeglass prescription values may be propagated from the last eyeglass prescription on file. In another embodiment, the initial eyeglass prescription values may be propagated from the auto refractor data. In a step S3204, an initial measurement for the sph value may be performed. If, while testing the initial measurement, the patient's vision is worse than 20/60, optometrist or doctor intervention may be required. In a step S3206, the axis value and the cyl value may be adjusted based on input from the patient. In one embodiment, to adjust the cyl, the following steps may be performed:

modifying the cyl to yield a first intermediate view and a second intermediate view;

comparing the first intermediate view with the second intermediate view;

determining via patient input that the first intermediate view is clearer than the second intermediate view;

adjusting the first intermediate view and the second intermediate view by an interim value; and

repeating said comparing, said determining, and said adjusting to yield an adjusted cyl.

In on embodiment, to adjust the axis, the following steps may be performed:

modifying the axis to yield a first intermediate view and a second intermediate view;

comparing the first intermediate view with the second intermediate view;

determining via patient input that the first intermediate view is clearer than the second intermediate view;

adjusting the first intermediate view and the second intermediate view by an interim value;

and

repeating said comparing, said determining, and said adjusting to yield an adjusted axis.

In a step S3208, the sph may be optimized according to input from the patient. In one embodiment, the sph may be optimized using the optimization at 20/40 module shown in FIG. 20, the optimization at 20/60 module shown in FIG. 21, the read 20/20 size module shown in FIG. 22, and the large size module shown in FIG. 23.

In a step S3210, the results of the automated refraction process may be output to a database. The results may be, for example, without limitation, a new eyeglass prescription of the patient. The optometrist or doctor may have access to the database to view the new eyeglass prescription of the patient.

What is claimed is:

1. An automated refraction process for determining an eyeglass prescription of a patient, executed by a processor, comprising:
obtaining patient information from the patient to generate initial sphere, cylinder, axis, add, and prism values;
performing measurements to generate at least one updated sphere, cylinder, axis, add, and prism value based on communication with the patient;
repeating the performing measurements to generate optimized sphere, cylinder, axis, add, and prism values; and
outputting the optimized sphere, cylinder, axis, add, and prism values to a database.

2. The process of claim 1, wherein performing measurements comprises:
performing sphere measurements, cylinder measurements, axis measurements, add measurements, and prism measurements to obtain the optimized sphere, cylinder, axis, add, and prism values.

3. The process of claim 1, further comprising:
sending an error report upon receiving an error; and
sending a completion report upon completion of the automated refraction process.

4. The process of claim 2, wherein the performing cylinder measurements comprises:
setting the initial cylinder value to a reference cylinder value;
applying the initial cylinder value to select a first optic and a second optic;
determining via a first patient input that the first optic is perceived by the patient to be clearer than the second optic, thus yielding an updated cylinder value from the initial cylinder value, while maintaining a spherical equivalent;
assigning the updated cylinder value to the initial cylinder value; and
repeating the applying, the determining, and the assigning to yield an intermediate cylinder value.

5. The process of claim 4, wherein the initial cylinder value is greater than a first threshold value.

6. The process of claim 5, further comprising:
generating a cylinder difference from the reference cylinder value and the intermediate cylinder value;
verifying that the cylinder difference is greater than a second threshold value;
selecting a third optic using the reference cylinder value and a fourth optic using the intermediate cylinder value;
determining via a second patient input whether the third optic is perceived by the patient to be clearer than the fourth optic; and
generating the optimized cylinder value based on a result of the determining, while maintaining the spherical equivalent.

7. The process of claim 2, wherein performing measurements to generate the updated axis value comprises:
setting the initial axis value to a reference axis value;
applying the initial axis value to select a fifth optic and a sixth optic;
determining via a third patient input that the fifth optic is perceived by the patient to be clearer than the sixth optic, thus yielding an updated axis value from the initial axis value;
assigning the updated axis value to the initial axis value; and
repeating the applying, the determining, and the assigning to yield an intermediate axis value.

8. The process of claim 7, further comprising:
generating an axis difference from the reference axis value and the intermediate axis value;
verifying that the axis difference is greater than a third threshold value;
selecting a seventh optic using the reference axis value and an eighth optic using the intermediate axis value;
determining via a fourth patient input whether the seventh optic is perceived by the patient to be clearer than the eighth optic; and
generating the optimized axis value based on a result of the determining.

9. The process of claim 2, wherein the performing sphere measurements comprises:
selecting a first letter size of a first set of letters to show the patient;
generating an updated sphere value by adjusting the initial sphere value to improve the patient's perception of the first set of letters; and
generating a tag value based on the first letter size.

10. The process of claim 9, further comprising:
selecting a second letter size based on the tag value;
displaying a line of a second set of letters of second letter size to the patient;
recording a response from the patient; and
adjusting the updated sphere value based on the response to generate an optimized sphere value.

11. The process of claim 10, wherein the first set of letters and second set of letters is a set of words or a set of images.

12. The process of claim 10, further comprising:
adjusting the optimized sphere value for a left eye and a right eye of the patient independently such that a first view presented to the left eye is visually identical to a second view presented to the right eye.

13. The process of claim 2, wherein the performing prism measurements comprises:
applying the initial prism value to select a ninth optic;
determining via a fifth patient input that the ninth optic is perceived by the patient to be unclear, thus yielding an updated prism value from the initial prism value;
assigning the updated prism value to the initial prism value; and
repeating the applying, the determining, and the assigning to yield an optimized prism value.

14. The process of claim 1, further comprising:
recording a baseline speaking time of the patient;
recording a speaking speed of the patient;
comparing the baseline speaking time with the speaking speed to determine a confidence level; and
utilizing the confidence level to calculate a correction rate during said performing.

15. The process of claim 2, wherein the performing add measurements comprises:
actuating a motor to set an automated reading rod into an active position to display a line of letters to the patient;
recording a response from the patient;
adjusting the updated add value based on the response to generate an optimized add value, wherein the updated add value is based on a reference chart and the reference chart is stored on the database; and
actuating the motor to set the automated reading rod into an inactive position.

16. The process of claim 1, wherein the initial sphere, cylinder, axis and prism values are chosen from the group consisting of a current eyeglass prescription of the patient, a last eyeglass prescription on file of the patient, and auto refractor data.

17. The process of claim 16, wherein the initial sphere value is greater than a threshold value and the threshold value is calculated based on the patient information.

18. The process of claim 2, further comprising:
communicating with the patient via a patient input device, wherein the patient input device is selected from a group consisting of a joystick, a keyboard, a touchscreen device, a camera, and a microphone.

19. The process of claim 18, wherein the communicating uses voice recognition to record a response from the patient.

20. A system comprising:
a processor; and
a memory that contains instructions that are readable by said processor to cause said processor to perform actions of:
obtaining patient information from the patient to generate initial sphere, cylinder, axis and prism values;
performing measurements to generate at least one updated sphere, cylinder, axis, and prism value based on communication with the patient;
repeating the performing measurements to generate optimized sphere, cylinder, axis, and prism values; and
outputting the optimized sphere, cylinder, axis, and prism values to a database.
